(19) United States
(12) Patent Application Publication
(10) Pub. No.: US 2006/0051824 A1
(43) Pub. Date: Mar. 9, 2006

(54) Tetrahydrocannabinoid antigens and method of use

(76) Inventors: Haoyun An, Carlsbad, CA (US); Junjie Xu, Hangzhou (CN); Jose Alberto Orlando Macall, San Diego, CA (US)

Correspondence Address: AZURE INSTITUTE;INTELLECTUAL PROPERTY DEPT., 4108 SORRENTO VALLEY BOULEVARD, SAN DIEGO, CA, 92124, US

(21) Appl. No.: 11/048,648

(22) Filed: Jan. 31, 2005

Related U.S. Application Data
(60) Provisional application No. 60/607,442, filed on Sep. 3, 2004.

(51) Int. Cl. G01N 33/537

(52) U.S. Cl. 435/7.92; 530/403

(57) ABSTRACT

The present invention is directed to Δ⁸-THC and Δ⁹-THC compounds useful for the covalent attachment to immunogenic molecules to form antigens for the preparation of specific binding molecules to Δ⁹-Tetrahydrocannabinol, Δ⁹-Tetrahydrocannabinoids, Δ⁸-Tetrahydrocannabinol, and Δ⁸-Tetrahydrocannabinoids, and their derivatives and metabolites. The present invention is directed to the compounds, their method of preparation, cell lines producing the specific binding molecules, methods of using the antigens to produce the specific binding molecules, and test devices containing the antigens, haptens, or specific binding molecules of the invention.

This application claims priority to U.S. provisional patent application Ser. No. 60/607,442, filed Sep. 3, 2004, which is hereby incorporated by reference in its entirety, including all Tables, Figures, and Claims.

### FIELD OF THE INVENTION

The present invention is directed to compounds and methods useful for generating antibodies.

### BACKGROUND OF THE INVENTION

The following Background of the Invention is intended to aid the reader in understanding the invention and is not admitted to be prior art.

Marijuana is a hallucinogen usually ingested by smoking the leaves of the Cannabus plant. It may also be orally ingested by eating products containing derivatives of the plant. After smoking or oral administration, the major psychoactive compound, Tetrahydrocannibinol (THC), is extensively metabolized before being excreted in the urine. THC is the active component in marijuana that provides the basis for its physiological activity, although other cannabinoids are also likely to be contributors to these effects. THC is rapidly absorbed by inhalation and through the gastrointestinal tract. It is almost completely metabolized over time in the body.

The detection of THC poses particular technical problems. THC itself is not detected when testing for marijuana use, since reliable means have not been available. Because THC is almost completely metabolized in the body, it is normally detected by determining the presence of its metabolites in urine or blood. However, for convenience and hygienic reasons, it would be more desirable to detect THC in saliva. THC found in saliva is present as the parent compound (THC) form since it has not yet been metabolized by the liver. But the antibodies used to detect THC metabolites in urine cannot detect the parent Δ⁸-THC and Δ⁹-THC compounds found in oral fluid, specifically Δ⁹-Tetrahydrocannabinol, a Δ⁹-Tetrahydrocannabinoid, Δ⁸-Tetrahydrocannabinol, and a Δ⁸-Tetrahydrocannabinoid. [See Cook, et al., NIDA Research Monograph 7, 15-27 (1976); Soares, et al. NIDA Research Monograph 42, 44-55 (1982); Gross, et al. J. Anal. Tox. 9, 1-5 (1985); Cook, et al. NIDA Research Monograph 42, 19-32 (1982); Owens, et al. NIDA Research Monograph 42, 33-43 (1982); U.S. Pat. Nos. 4,833,073; 5,237,057; 5,302,703; 5,635,530; 4,391,904; 5,219,747; and European patent numbers 0,276,732 and 0,736,529 (1996)]. Additionally, the parent compound forms of THC are very labile, and also stick to the sides of containers, thereby significantly reducing their concentration in a short time. There is thus an urgent need for better compounds and methods for detecting THC.

### SUMMARY OF THE INVENTION

The present invention is involves novel antigens, which are useful for the preparation of antibodies directed to Δ⁸- and Δ⁹-THC parent compounds and their metabolites. In particular, the Δ⁸- and Δ⁹-THC parent compounds are found in the saliva of persons ingesting THC-containing substances, such as marijuana. Thus, the present invention relates to compositions and methods of using them to determine the presence of THC in saliva. The present invention provides methods of making the antigens, and methods of preparing antibodies that are generated by the use of the antigens and which are directed to Δ⁸- and Δ⁹-THC compounds. The compounds of the present invention can function as haptens and produce antibodies that bind to Δ⁹-Tetrahydrocannabinol, Δ⁹-Tetrahydrocannabinoid, Δ⁸-Tetrahydrocannabinol, and Δ⁸-Tetrahydrocannabinoid. In some embodiments the antibodies bind specifically to these molecules. The resulting antibodies can be used to design sensitive, easy, convenient, non-invasive, fast, and reliable immunoassays for detecting the predominant Δ⁹- and Δ⁸-THC components of marijuana in oral fluid or saliva.

In a first aspect the present invention provides compounds having the chemical formula: 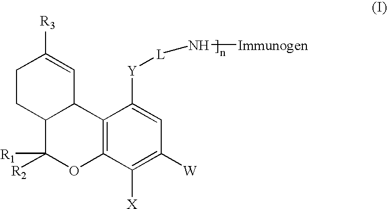
wherein

R₁, R₂, and R₃ are independently selected from H, C₁₋₃ alkyl, C₁₋₃ alkoxy, C₁₋₃ thioalkyl, CN, COOH, CH₂OH, and NO₂;

Y is selected from O, S, C═O, —CH₂—, —CH₂NH—, and C═N;

W is selected from C₂₋₈ branched or straight chain alkyl;

X is H or C₁₋₃ alkyl;

n is an integer of from 1 to 100;

L is a linker molecule selected from CH₂(CH₂)_{1,3-5}CO, (CH₂)₀₋₅CH═CHCO, CH₂(C₆H₄)_{z}CO, COCH₂(CH₂)₂₋₅CO, COCH₂(CH₂)₀₋₅OCH₂(CH₂)₀₋₅CO, and CH₂(CH₂)₀₋₅NHCO(CH₂)₀₋₅S; where z indicates an ortho-, meta-, or para-substitution.

Ranges of numbers used herein are intended to convey any individual number or sub-range of numbers within the specified range. Thus, 0-5 indicates any one of 0, 1, 2, 3, 4, and 5, as if each number was set forth specifically herein, and also includes, for example, 0-1, 1-3, 2-5, 0 and 2-5, 3-5, etc.

“Immunogens” are molecules that interact with the animal body's immune system and stimulate the production of antibodies in the animal body. Immunogens are covalently conjugated with haptens. Suitable immunogens include, for example, proteins, natural and synthetic polypeptides, and polysaccharides. Examples of immunogens include Keyhole limpet hemocyanin (KLH), Bovine gamma globulin (BGG), Bovine Serum Albumin (BSA), Bovine Thyroglobulin (BTG), Hen egg-white Lysozyme (HEL), Ovalbumin (OVA), Sperm Whale Myoglobin (SWM), Tetanus Toxoid (TT), Methylated Bovine Serum Albumin (mBSA), Rabbit Serum Albumin (RSA), Human immunoglobulins IgG and IgA, agarose-based gel filtration matrixes, beads, or other such compounds known in the art. In various embodiments, any number of from 1-100 or from 10-80 of the THC-derivative hapten molecules may be linked to an immunogen molecule. The term “hapten” refers to a small molecule that contains an immunogenic determinant, but which is not itself antigenic unless combined with an antigenic carrier or immunogen. Examples of haptens include Δ⁹-THC, Δ⁸-THC, their metabolites produced in a biological organism, or their derivatives by chemical modification. The immunogenic determinant is the molecular structure against which it is desired to form antibodies.

In some embodiments when Y is O, then L is not CH₂(CH₂)_{0,2}CO; and when Y is O and the Immunogen is human serum albumin, then L is not COCH₂(CH₂)CO; and when L is COCH₂CH₂CO, then Immunogen is not human serum albumin. “Alkyl” refers to a saturated aliphatic hydrocarbon including straight chain and branched chain groups. As an example of terminology, CH₂(CH₂)_{0,2}CO refers to the formula CH₂(CH₂)ₓCO where the x can be 0 or 2. Similar formulae are interpreted in a similar manner. “Alkoxy” refers to an -0-alkyl and an —O— cycloalkyl group. Examples of alkoxy include methoxy or trihalomethoxy. “Thioalkyl” refers to S-alkyl, where “S” is a sulfur joined to an alkyl.

In one embodiment the compounds have the chemical formula: 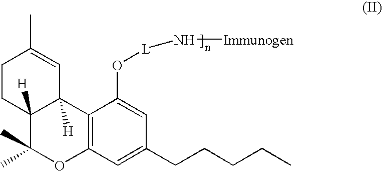
wherein

L is a linker molecule selected from CH₂(CH₂)_{1,3-5}CO, (CH₂)₀₋₅CH═CHCO, CH₂(C₆H₄)_{z}CO, COCH₂(CH₂)₂₋₅CO, COCH₂(CH₂)0-5OCH₂(CH₂)₀₋₅CO; CH₂(CH₂)₀₋₅NHCO(CH₂)₀₋₅S. N is any integer from 1 to 100 and z indicates an ortho-, meta-, or para-substitution. The immunogen can be keyhold limpet hemocyanin (KLH), bovine gamma globulin (BGG), bovine serum albumin (BSA), bovine thyroglobulin (BTG), hen egg-white lysozyme (HEL), ovalbumin (VA), sperm whate myoglobin (SWM), tetranus toxoid (TT), flagellin, human IgG, or an agarose particle. Again, as used herein, ranges of numbers are intended to convey each number within the specified range as if specifically and individually set forth herein. Thus, 0-5 indicates 0, 1, 2, 3, 4, and 5 and all sub-ranges therein.

In another embodiment L is selected from CH₂(CH₂)_{1,3}CO, (CH₂)₁₋₂CH═CHCO, CH₂(C₆H₄)_{z}CO, COCH₂(CH₂)₂₋₅CO, COCH₂(CH₂)₁₋₂OCH₂(CH₂)₁₋₂CO, and CH₂(CH₂)₁₋₃NHCO(CH₂)₁₋₃S, where z indicates ortho-, meta-, or para, and n is any integer of from 10 to 80.

In various embodiments the compound can be any of the following:

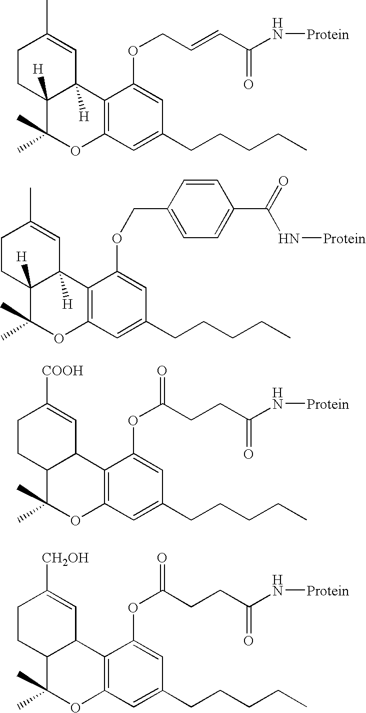

The terms “linking group,” “linker molecule,” and “linker” refer to the molecular group linking the hapten and the immunogen. In order to perform the linkage between the hapten and the immunogen, it is necessary that each of the reactants contain a chemically complementary group. Examples of complementary groups are amino and carboxyl to form amide linkages, or carboxy and hydroxy to form ester linkages, or amino and alkyl halides to form alkylamino linkages, or thiols and thiols to form disulfides, or thiols and maleimides or alkylhalides to form thioethers. Hydroxyl, carboxyl, amino and other functionalities may be introduced by known methods when not already present. Likewise, a wide variety of linking groups may be employed. The structure of the linkage should be a stable covalent linkage formed to attach the hapten or derivative thereof to the immunogen, which can be a protein, polypeptide, or other immunogen. The covalent linkages should be stable relative to the solution conditions under which the ligand and linking group are subjected. Generally, linking groups will be from 1-20 carbons and 0-10 heteroatoms (NH, O, S) and may be branched or straight chain. But in other embodiments the linking groups can be from 10-80 carbons or 20-70 carbons or 20-50 carbons or 10-20 carbons or 10 to 30 carbons, all having from 0-10 heteroatoms. Combinations of atoms which are chemically compatible comprise the linking group. For example, amide, ester, thioether [C—S—C], thioester [—S—C(O)—], keto, hydroxyl, carboxyl, ether groups in combinations with carbon--carbon bonds are acceptable examples of chemically compatible linking groups. Other chemically compatible compounds which may comprise the linking group are set forth in this disclosure.

In another aspect the present invention provides methods of producing antibodies. The methods involve administering to a host animal a compound of Structure I, wherein R₁, R₂, and R₃ are independently selected from H, C₁₋₃ alkyl, C₁₋₃ alkoxy, C₁₋₃ thioalkyl, CN, COOH, CH₂OH, or NO₂;

Y is O, S, C═O, —CH₂—, —CH₂NH—, or C═N;

W is C₂₋₈ branched or straight chain alkyl;

X is H or C₁₋₃ alkyl;

L is a linker molecule selected from CH₂(CH₂)₁₋₅CO, (CH₂)₀₋₅CH═CHCO, CH₂(C₆H₄)_{z}CO, COCH₂(CH₂)_{0,2-5}CO, COCH₂(CH₂)₀₋₅OCH₂(CH₂)₀₋₅CO; CH₂(CH₂)₀₋₅NHCO(CH₂)₀₋₅S;

where n is an integer of from 1 to 100, z indicates an ortho-, meta-, or para-substitution;

and producing the antibodies.

In one embodiment the methods involve using a compound having the formula of Structure II where L is a linker molecule selected from CH₂(CH₂)₀₋₅CO, CH₂(CH₂)₁₋₅CO, (CH₂)₀₋₅CH═CHCO, CH₂(C₆H₄)_{z}CO, COCH₂(CH₂)₀₋₅CO, COCH₂(CH₂)_{0,2-5}CO, COCH₂(CH₂)₀₋₅OCH₂(CH₂)ₘCO; CH₂(CH₂)₀₋₅NHCO(CH₂)₀₋₅S, and n is an integer from 1 to 100. In another embodiment the linker is selected from CH₂(CH₂)₀₋₃CO, (CH₂)₁₋₂CH═CHCO, CH₂(C₆H₄)_{z}CO, COCH₂(CH₂)₂₋₅CO, COCH₂(CH₂)₁₋₂OCH₂(CH₂)₁₋₂CO; CH₂(CH₂)₁₋₃NHCO(CH₂)₁₋₃S, and n is an integer of from 10 to 80. “Host animals” include any animal whose immune system will generate antibodies to the hapten in response to exposure. Examples of host animals include mice, rats, rabbits, goats, sheep, cattle, horses and chickens. Exposure will normally occur by injecting the hapten into the animal in the form of an immunogen. Host animals can also be a mammal, or a rodent, or a lagomorph.

In various embodiments the compounds used in the methods can be one or more of the following:

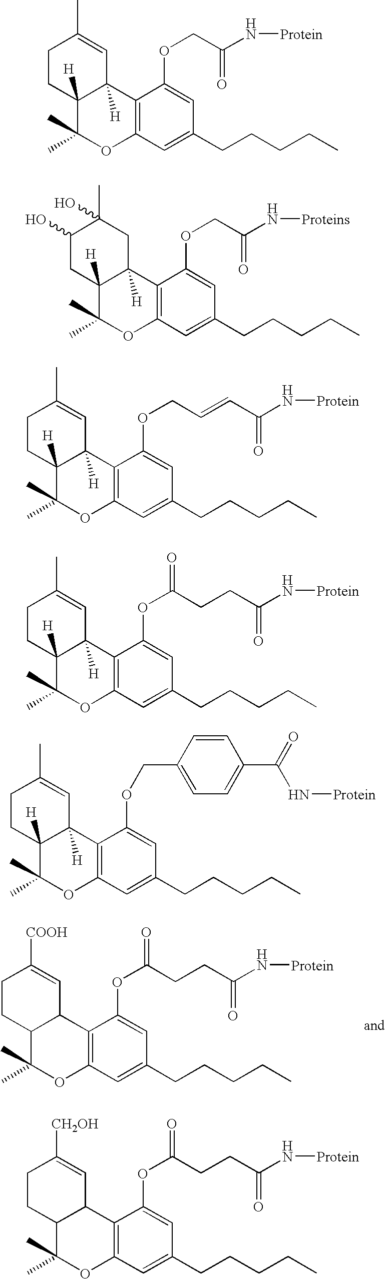

In another aspect the present invention provides compounds of the formula: 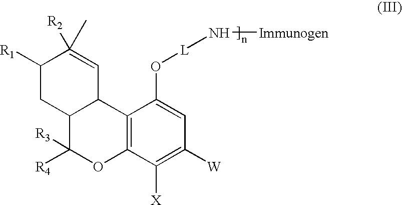
wherein

R₁ and R₂ are independently H, OH, NH₂, O-alkyl, COOH, CONH₂, CN, SH, and S-alkyl;

R₃ and R₄ are independently H, OH, NH₂, O-alkyl, COOH, CONH₂, CN, SH, C₁₋₃ alkyl, C₂₋₃ alkenyl, or S-alkyl;

X is H or C₁₋₃ alkyl;

W is a C₂₋₈ branched or straight chain alkyl;

L is a linker molecule selected from CH₂(CH₂)₀₋₅CO, (CH₂)₀₋₅CH═CHCO, CH₂(C₆H₄)_{z}CO, COCH₂(CH₂)₀₋₅CO, COCH₂(CH₂)₀₋₅OCH₂(CH₂)₀₋₅CO, CH₂(CH₂)₀₋₅NHCO(CH₂)₀₋₅S where z indicates ortho-, meta-, or para-; and n is an integer from 1 to 100.

In various embodiments the compound has the formula of Structure III where R₁ and R₂ are independently OH, H, NH₂, O-alkyl, and SH;

R₃ and R₄ are independently OH, H, NH₂, O-alkyl, C₁₋₃ alkyl, C₂₋₃ alkenyl, or SH. L is a linker molecule selected from CH₂(CH₂)₀₋₃CO, (CH₂)₁₋₂CH═CHCO, CH₂(C₆H₄)ₓCO, COCH₂(CH₂)₁₋₅CO, COCH₂(CH₂)₁₋₂OCH₂(CH₂)₁₋₂CO; CH₂(CH₂)₁₋₃NHCO(CH₂)₁₋₃S, and n can be any number from 1-100 or any number from 10-80.

In one embodiment the compound has the structure:

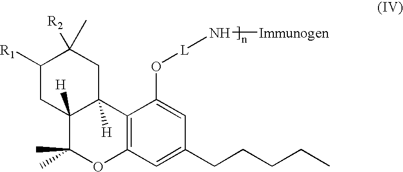

where R₁ and R₂ are independently selected from OH, H, NH₂, O-alkyl, or SH, and L is a linker molecule selected from CH₂(CH₂)₀₋₃CO, (CH₂)₁₋₂CH═CHCO, CH₂(C₆H₄)ₓCO, COCH₂(CH₂)₁₋₅CO, COCH₂(CH₂)₁₋₂OCH₂(CH₂)₁₋₂CO; CH₂(CH₂)₁₋₃NHCO(CH₂)₁₋₃S, and the immunogen is as described herein. In one embodiment R₁ and R₂ are each OH.

In another embodiment of the invention the compound has the formula:

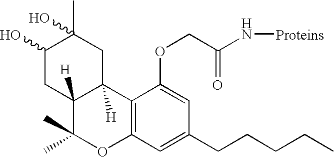

In another aspect the invention provides methods of producing specific binding molecules involving administering to a host animal a compound of Structure III where

R₁ and R₂ are independently selected from OH, H, NH₂, O-alkyl, COOH, CONH₂, CN, SH, and S-alkyl;

R₃ and R₄ are independently selected from H, O-alkyl, CONH₂, CN, C₁₋₃ alkyl, and C₂₋₃ alkenyl;

L is a linker molecule selected from CH₂(CH₂)₀₋₅CO, (CH₂)₀₋₅CH═CHCO, CH₂(C₆H₄)_{z}CO, COCH₂(CH₂)₀₋₅CO, COCH₂(CH₂)₀₋₅OCH₂(CH₂)₀₋₅CO; CH₂(CH₂)₀₋₅NHCO(CH₂)₀₋₅S, n is any integer from 1 to 100 or any integer of from 10 to 80. z indicates an ortho-, meta-, or para-substitution.

In another embodiment of the methods the compound has the formula of Structure IV where

R₁ and R₂ are selected from OH, H, NH₂, O-alkyl, and SH;

L is a linker molecule selected from CH₂(CH₂)₀₋₃CO, (CH₂)₁₋₂CH═CHCO, CH₂(C₆H₄)ₓCO, COCH₂(CH₂)₁₋₅CO, COCH₂(CH₂)₁₋₂OCH₂(CH₂)₁₋₂CO; CH₂(CH₂)₁₋₃NHCO(CH₂)₁₋₃S; and the immunogen is selected from Keyhold limpet hemocyanin (KLH), bovine gamma globulin (BGG), bovine serum albumin (BSA), bovine thyroglobulin (BTG), hen egg-white lysozyme (HEL), ovalbumin (VA), sperm whate myoglobin (SWM), tetranus toxoid (TT), flagelin, human IgG, and an agarose particle. In various embodiments n is any integer of from 1-100 or any integer of from 10-80. In one embodiment R₁ and R₂ are each OH.

In various embodiments of the methods the specific binding molecules bind specifically to any one or more of Δ⁹-Tetrahydrocannabinol, a Δ⁹-Tetrahydrocannabinoid, Δ8-Tetrahydrocannabinol, a Δ⁸-Tetrahydrocannabinoid, and metabolites thereof. In another embodiment the specific binding molecules bind to all of these. In one embodiment the specific binding molecules are antibody molecules.

In other methods of the invention, any compound described herein (supra. or infra.) is administered to a host animal, and antibodies are thereby produced. The methods can further involve harvesting the antibodies from the host animals, and purifying the antibodies. The purification can be done by any method, such as column chromatography or affinity chromatography, or any convenient method.

In another aspect, the invention provides compounds of the formula:

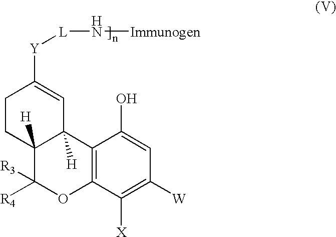

where Y is selected from —C═O, —CH₂—, —CH₂NH—, or —C═N—;

L is a linker molecule selected from no linker (null), NHCH₂(CH₂)₀₋₃CO, O(CH₂)₁₋₃CO, NH(CH₂)₂₋₃S(CH₂)₂₋₃CO, NH(CH₂)₂₋₅NHCO(CH₂)₁₋₅CO, CO(CH₂)₁₋₅CO, (CH₂)₁₋₅CO, (CH₂)₁₋₅NHCO(CH₂)₁₋₅CO, NHCH(COOH)CH₂S, and (CH₂)S(CH₂)₁₋₅CO;

R₁ and R₂ are independently selected from OH, H, NH₂, O-alkyl, COOH, CONH₂, CN, SH, or S-alkyl;

R₃ and R₄ are independently selected from H, COOH, C₁₋₃ alkyl, C₂₋₃ alkenyl, O-alkyl, CONH₂, CN, and S-alkyl;

W is a C₂₋₈ branched or straight chain alkyl;

X is H or C₁₋₃ alkyl; and n is an integer from 1 to 100.

In one embodiment the compound has the formula: 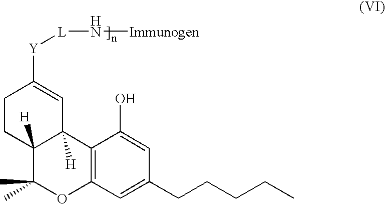
where L is a linker molecule selected from the above,
n is an integer from 1 to 100 or from 10-80. The immunogens are as described herein.

In various embodiment the compound be any of the following:

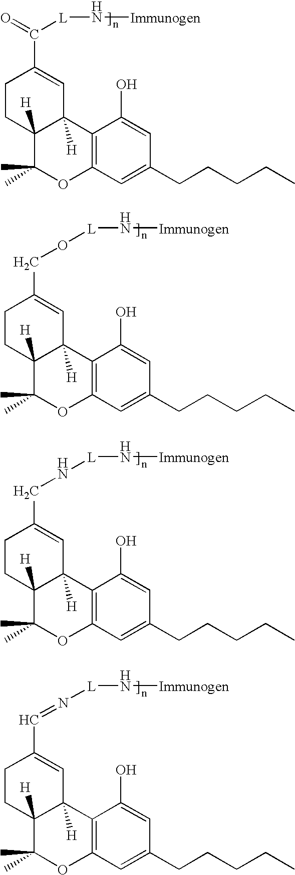

In another aspect the invention provides a compound of the formula:

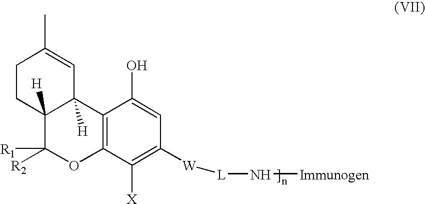

wherein R₁ and R₂ are independently selected from H, C₁₋₃ alkyl, C₁₋₃ alkoxy, C₁₋₃ thioalkyl, CN, and NO₂;

X is H or C₁₋₃ alkyl;

W is a C₂₋₈ branched or straight chain alkyl; and

L is selected from none, CO, (CH₂)₁₋₅CO, NH(CH₂)₁₋₅CO, HNCO(CH2)₀₋₃CH₂CO, NHCO(CH₂)₁₋₃CO, O(CH₂)₁₋₃CO, OCO(CH₂)₁₋₃CO, and (CO(CH₂)₁₋₃O(CH₂)₁₋₃CO;

In one embodiments if the immunogen is bovine serum albumin and W is (CH₂)₅, then L is not CO.

In another embodiment the compound has the formula:

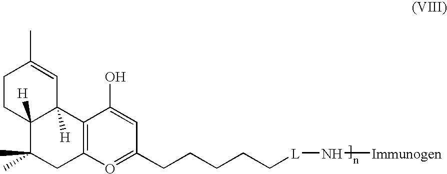

wherein L is selected from none, CO, (CH₂)₁₋₅CO, NH(CH₂)₁₋₅CO, HNCO(CH₂)₀₋₃CH₂CO, NHCO(CH₂)₁₋₃CO, O(CH₂)₁₋₃CO, OCO(CH₂)₁₋₃CO, and (CO(CH₂)₁₋₃O(CH₂)₁₋₃CO. n can be any integer from 1-100 or from 10-80. In one embodiment L is not CO if Immunogen is bovine serum albumin.

In another aspect the present invention provides methods of synthesizing an antigen. The methods involve contacting Δ⁹-tetrahydrocannabinol with a bromoacetate to product a 1-ether; hydrolyzing the ester to product a carboxylic acid derivative; activating the carboxylic acid derivative to produce an N-succinamyl ester; and conjugating the N-succinamyl ester to synthesize an antigen. In one embodiment the activation is performed by contacting the carboxylic acid derivative with N-hydroxysuccinamide and 1 -ethyl-3-(dimethylpropylamino)carbodiimide hydrochloride. “Antigens” are immunogenic proteins or polymers having the property of independently eliciting an immunogenic response in a host animal and that can be covalently coupled to a hapten. An antigen will typically include a hapten and an immunogen.

In another embodiment the method of synthesizing an antigenic compound involves the steps of:

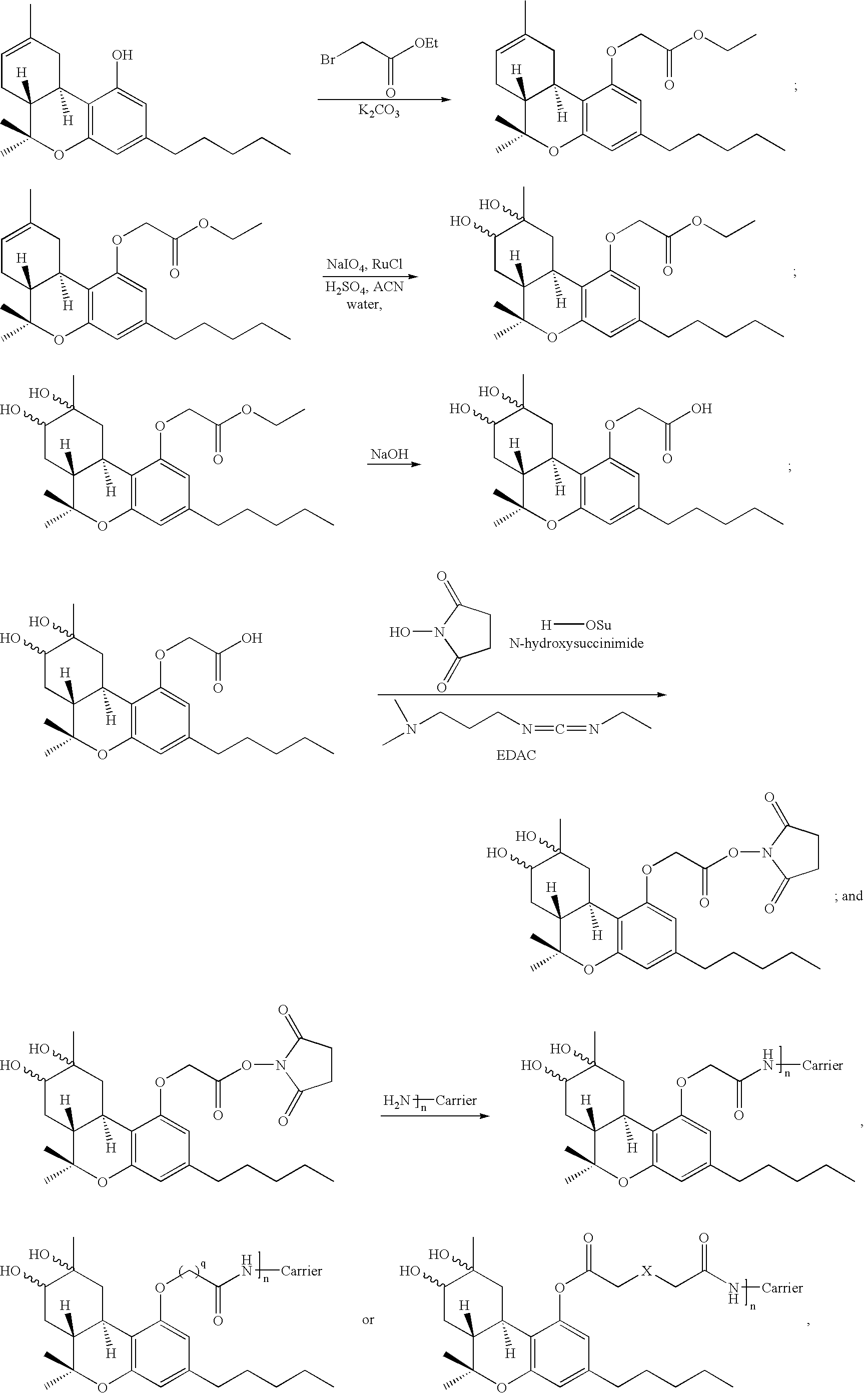

wherein q is 1-4 and X is none, O, or CH₂ and n is any integer of from 1-100 or from 10-80.

In another aspect the present invention provides specific binding molecules produced according to any of the methods disclosed herein. The specific binding molecule can specifically bind to any one or more of Δ⁹-Tetrahydrocannabinol, a Δ⁹-Tetrahydrocannabinoid, Δ⁸-Tetrahydrocannabinol, and a Δ⁸-Tetrahydrocannabinoid, or their derivatives. In another embodiment the specific binding molecule binds to any one or more of the compounds described herein. In one embodiment the specific binding molecules bind to any one or more of the compounds described herein and numbered 1-55 in the specification and Figures. In one embodiment the specific binding molecule is an antibody or a fragment of an antibody. “Antibody” refers to an immunoglobulin, whether natural or partially or wholly synthetically produced. The term also includes derivatives thereof which maintain specific binding ability. The term also covers any protein having a binding domain which is homologous or largely homologous to an immunoglobulin binding domain. These proteins may be derived from natural sources, or partly or wholly synthetically produced. An antibody may be monoclonal or polyclonal, and can be a member of any immunoglobulin class (or combination of classes), including any of the human classes: IgG, IgM, IgA, IgD, IgG, and IgE. An “antibody fragment” is any derivative of an antibody which is less than full-length. The antibody fragment can retain at least a significant portion of the full-length antibody's specific binding ability. Examples of antibody fragments include, but are not limited to, Fab, Fab′, F(ab′)₂, scFv, Fv, dsFv diabody, and Fd fragments. A “derivative” is any molecule having the same basic structure as the parent compound. In one embodiment, derivatives are compounds 1-54 as described in the specification and Figures, except for Δ⁹-Tetrahydrocannabinol, a Δ⁹-Tetrahydrocannabinoid, Δ8-Tetrahydrocannabinol, and a Δ⁸-Tetrahydrocannabinoid, which are parent compounds. The invention also provides a cell line that produces specific binding molecules that bind to any of the compounds described herein.

The antibody fragment may be produced by any means. For instance, the antibody fragment may be enzymatically or chemically produced by fragmentation of an intact antibody or it may be recombinantly produced from a gene encoding the partial antibody sequence. Alternatively, the antibody fragment may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

Single-chain Fvs (scFvs) are recombinant antibody fragments consisting of only the variable light chain (V_{L}) and variable heavy chain (V_{H}) covalently connected to one another by a polypeptide linker. Either V_{L} or V_{H} may be the NH₂-terminal domain. The polypeptide linker may be of variable length and composition so long as the two variable domains are bridged without serious steric interference. Typically, the linkers are comprised primarily of stretches of glycine and serine residues with some glutamic acid or lysine residues interspersed for solubility. “Diabodies” are dimeric scFvs. The components of diabodies typically have shorter peptide linkers than most scFvs and they show a preference for associating as dimers.

An “Fv” fragment consists of one V_{H} and one V_{L} domain held together by noncovalent interactions. The term “dsFv” is used herein to refer to an Fv with an engineered intermolecular disulfide bond to stabilize the V_{H}-V_{L} pair. A F(ab′)₂ fragment is an antibody fragment essentially equivalent to that obtained from immunoglobulins (typically IgG) by digestion with an enzyme pepsin at pH 4.0-4.5. The fragment may be recombinantly produced. A Fab′ fragment is an antibody fragment essentially equivalent to that obtained by reduction of the disulfide bridge or bridges joining the two heavy chain pieces in the F(ab′)₂ fragment. The Fab′ fragment may be recombinantly produced. A “Fab” fragment is an antibody fragment essentially equivalent to that obtained by digestion of immunoglobulins (typically IgG) with the enzyme papain. The Fab fragment may be recombinantly produced. The heavy chain segment of the Fab fragment is the Fd piece.

Active fragments of antibodies preferably include the Fv region of an antibody. Active fragments of antibodies can be made using methods known in the art, such as proteolytic digestion of samples including antibodies. Antibodies may be polyclonal or monoclonal, unless otherwise specified. A preparation of antibodies can be crude, such a whole blood or serum or plasma, or can be partially purified, such as by crude separation methods such as molecular weight purification or ammonium sulfate precipitation, or can be substantially purified, such as by affinity chromatography for a class of antibody, subclass of antibody, or by binding with a particular antigen or epitope. Methods for such purification are known in the art, such as provided by Harlow and Lane, Antibodies, A Laboratory Manual, Cold Spring Harbor (1988).

A further aspect of the present invention is a compound having any of the wing chemical formulae:

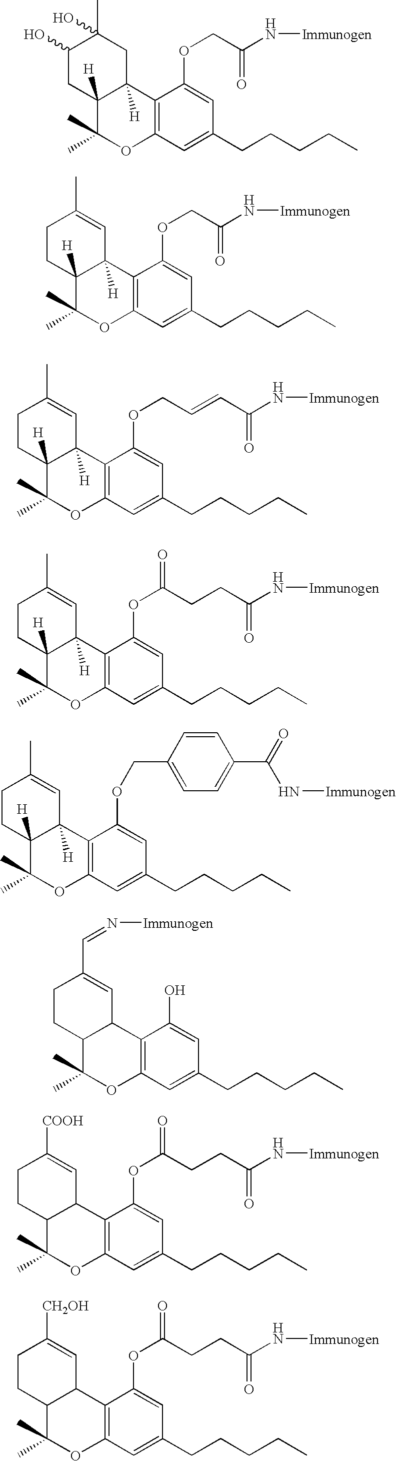

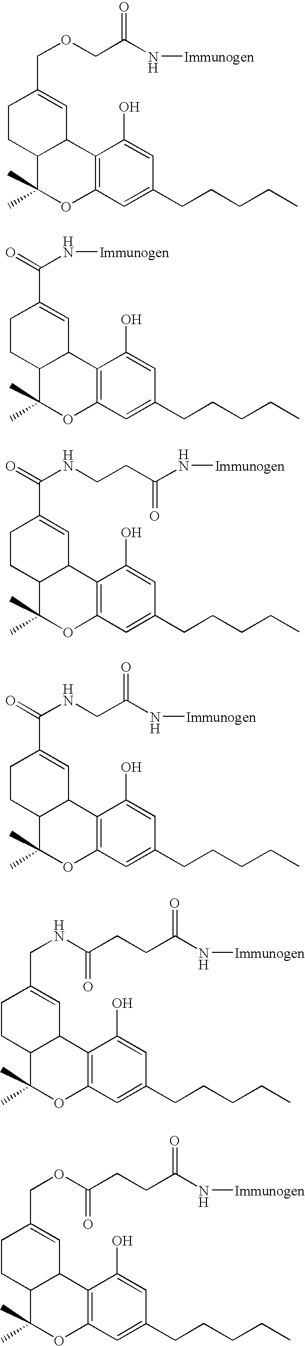

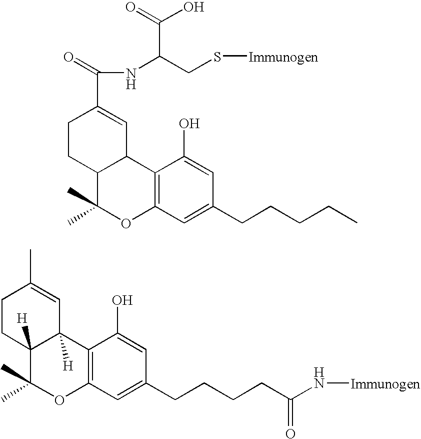

The Immunogen can be any immunogen. In one embodiment the immunogen is a protein or proteinaceous molecule. In various embodiments the protein is any of keyhole limpet hemocyanin, bovine gamma globulin, bovine serum albumin, bovine thyroglobulin, hen egg-white lysozyme, ovalbumin, sperm whale myoglobin, tetanus toxoid, methylated bovine serum albumin, rabbit serum albumin, human IgG, human IgA. The “immunogen” can also be an agarose-containing gel filtration matrix.

The present invention includes a variety of other useful aspects, which are detailed herein. These aspects of the invention utilize the articles of manufacture and compositions of matter described herein.

The summary of the invention described above is not limiting and other features and advantages of the invention will be apparent from the following detailed description, as well as from the claims.

### BRIEF DESCRIPTION OF THE DRAWINGS

FIG. 1 illustrates a primary active constituents of marijuana, Δ9-tetrahydrocannabinol (THC) and metabolites.

FIG. 2 illustrates the conjugation sites available on the core THC molecule. Conjugation involves covalently bonding a haptenic compound to the immunogenic carrier for the formation of an antigen, which is used to induce production of antibodies in a host animal.

FIG. 3 illustrates synthesis of Δ⁹-THC-CH₂CO antigens using conjugation at position O¹.

FIG. 4 illustrates synthesis of dihydroxy-THC antigens using conjugation at position O¹.

FIG. 5 illustrates synthesis of Δ⁹-THC-CH₂CH═CHCO antigens using conjugation at position O¹.

FIG. 6 illustrates synthesis of Δ⁹-THC-ester [R₁C(O)OR₂] antigens using conjugation at position O¹.

FIG. 7 illustrates synthesis of Δ⁹-THC-CH₂C₆H₄CO antigens using conjugation at position O¹.

FIG. 8 illustrates synthesis of optically pure 11-nor-Δ⁹-THC-9-carboxylic acid and related haptens.

FIG. 9 illustrates synthesis of Δ⁹-THC-9 antigens using conjugation at position 9.

FIG. 10 illustrates synthesis of Δ⁹-THC-9-CH₂O antigens using conjugation at the 9 position.

FIG. 11 illustrates the binding affinity of control antigens versus Δ⁹-THC test antigens of the present invention to a control antibody that binds Δ⁸-THC (AF9-14FR-4-2).

FIG. 12 illustrates the competitive inhibition of Δ⁸-THC for the Δ⁸-THC antibody (AF9-14FR-4-2) by novel Δ⁹-THC test antigens of the present invention.

### DETAILED DESCRIPTION

In the following detailed description, reference is made to the accompanying drawings that form a part hereof, and in which is shown by way of illustration specific embodiments in which the invention may be practiced. It is understood that other embodiments may be utilized and structural changes may be made without departing from the scope of the present invention.

The present invention is directed to novel cannabinoid compounds, particularly Δ8- and Δ⁹-THC and their derivatives, as haptenic compounds to be covalently conjugated or bonded to a conventional immunogenic carrier material through various spacers/linkers to provide novel antigens or immunogens. Linkers are structural fragments that are used to bond or conjugate the haptens onto an immunogenic carrier. In one embodiment the linkers are first covalently bonded to haptens through any of a variety of chemical approaches, and then the other end of the linker is activated and reacted with the amino groups on the carrier.

The immunogens of the present invention induce the formation of antibodies specific to Δ⁹-/Δ⁸-THC compounds in host animals by injection of the immunogen into a host animal. Suitable host animals include (but are not limited to) mammals such as rabbits, rats, mice, horses, goats, sheep, cows, etc., or alternatively fowl, such as chickens. The resulting antisera contains antibodies that selectively bind to Δ⁹-/Δ⁸-THC and their derivatives.

The present invention also involves the use of the novel antigens described herein for the induction of specific binding molecules. The specific binding molecules produced recognize Δ⁹- and Δ⁸-THC. Therefore, the methods and devices of the invention are useful for detecting the parent compounds, Δ⁹- and Δ⁸-THC, as well as their metabolites and derivatives. THC and its metabolites can be detected in various body fluids, such as oral fluid, saliva, urine, and blood. A “specific binding molecule” refers to a molecule that binds to a target analyte (e.g., THC or its derivatives) and does not substantially bind to any other molecule present in the sample. In some embodiments a specific binding molecule can also bind to a molecule that correlates with or indicates the presence of an analyte of interest in a sample. By substantial binding is meant that binding occurs to an extent that will affect the result of an assay performed with the specific binding molecules, i.e., a less optimal or less accurate result will be obtained. A small amount of non-specific binding that may occur and that does not change the result of the assay is not considered substantial binding. In some embodiments the specific binding molecule can be an antibody or an antibody fragment (e.g., the Fab region of an antibody), an antigen, a receptor or fragment of a receptor that binds a ligand, or a member of a biotin-streptavidin pair or other type of binding pair. A derivative is a chemical substance related structurally to another substance and theoretically derivable from it. A derivative of a substance can be made from another substance without an unreasonable number of steps. As an example, the compounds numbered 1-55 in the Figures are all derivatives of Δ⁸or Δ⁹ THC parent compounds. Of course many other compounds are also derivatives of these parent compounds, as will be understood by those of ordinary skill in the art.

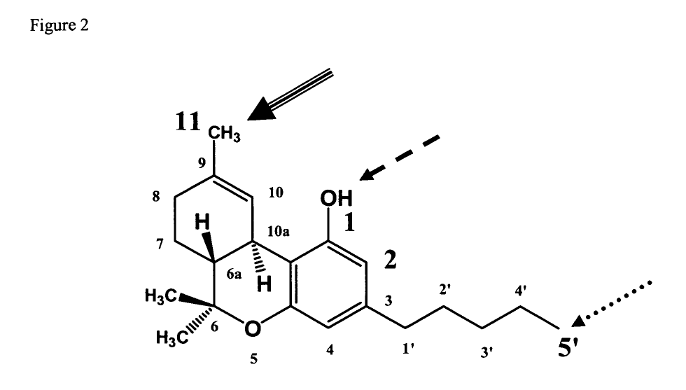

In different embodiments of the invention, positions 1, 9 and 5′ of the THC molecule are selected as conjugation sites (see FIG. 2) for the linker and immunogen. Conjugation of linker and immunogen at these sites does not adversely affect the inductive power of the attached immunogen. Different spacers can be used for conjugation at different sites.

Previously, testing of individuals for the use of marijuana has revolved around detecting the appearance of THC metabolites in the urine of the using individual. However, the present invention allows the detection of the parent Δ⁸ and Δ⁹ compounds in the saliva. Without wanting to be bound by any particular theory, it is believed that the creation of antibodies that bind to the parent compounds becomes possible by using haptens bound to immunogens and also using a linking molecule of precise length. Using the present invention, antibodies are generated to the parent Δ⁸ and Δ⁹ compounds of THC, making it possible to detect the parent THC compounds in saliva and oral fluid. Furthermore, higher yields of immunogenic compositions are obtainable using the present invention, therefore resulting in a lower cost of producing the compositions.

After synthesis of the specific binding molecules of the invention, they can be incorporated in any of a variety of assays for the detection of THC. In one embodiment the specific binding molecules of the present invention are isolated from the body fluid of a host animal. The specific binding molecules may then be purified from the body fluid.

The invention also provides cell lines for producing the specific binding molecules of the invention. In one embodiment the cell line is a hybridoma cell line. The cell lines can produce native specific binding molecules, or can also be engineered to produce chimeric specific binding molecules. The cell lines producing the specific binding molecules can be prepared using methods known to those of ordinary skill in the art. For example, when mice are the host animal, the mice can be immunized with a composition of the present invention. Spleen cells from the host animal can then be fused with a murine myeloma cell line, and the cells distributed on a culture plate. Culture supernatant from the cell line. When a chimeric specific binding molecule is desired, it can be produced by using a suitable expression vector and other techniques known to those of ordinary skill in the art.

Synthesis of Exemplary Compounds

The following experimental procedures are provided as examples of the synthesis and use of the compounds and immunogens described herein. However, it should be appreciated that various modifications may be made without departing from the inventive concepts presented herein.

### EXAMPLE 1

### Synthesis of Δ⁹-THC-CH₂CO Antigens

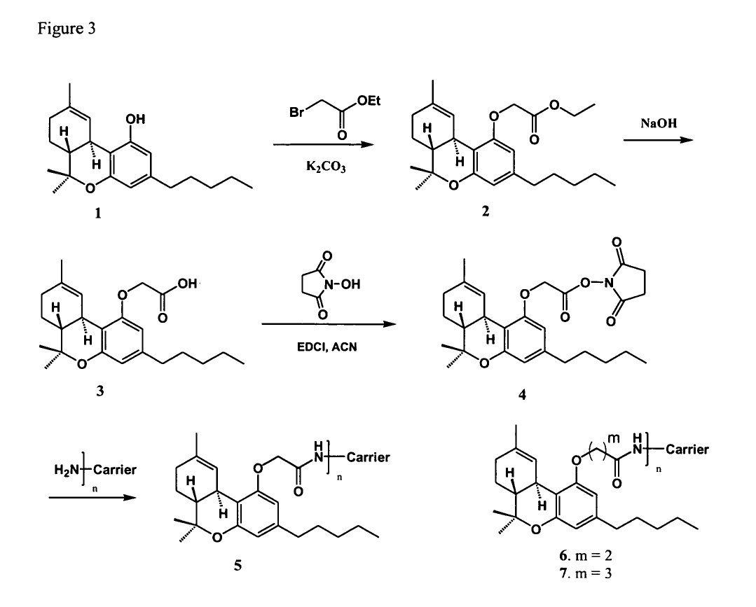

Referring to FIG. 3, Δ⁹-THC (1) was reacted with ethyl alpha-bromoacetate under carbonate basic condition to provide the corresponding 1-ether 2. Further hydrolysis of the ester provided the corresponding carboxylic acid derivative 3, which was further activated with N-hydroxysuccinamide and 1-ethyl-3-(dimethylpropylamino)carbodiimide (EDC) hydrochloride. The N-succinamyl ester 4 was used directly, without purification, for conjugation onto different carriers to give the desired compound 5. Compounds 6-8, which have different spacers, were synthesized from the different halo-esters by a similar strategy. The resulting compounds were purified on a G-50 SEPHADEX® gel filtration column. The purification was monitored by absorbance at 280 nm. The antigens were also purified by dialysis with PBS buffer.

Further details of this preparation procedure are provided below:

1-O-Ethyloxycarbonylmethyl-Δ⁹-THC (2).

An ethanol solution of Δ⁹-THC (1) (100 mg, 0.318 mmol) was transferred to a round bottom flask (see FIG. 3). The original vial was washed with methanol and the washes added to the flask. The solvent was then removed on the rotary evaporator, resulting in a yellow oil. The isolated Δ⁹-THC yellow oil was dissolved in 10 mL of acetone (dried over K₂CO₃) followed by the addition of potassium carbonate(0.45 g, 3.2 mmol). 71 uL of ethyl bromoacetate (107 mg, 0.64 mmol, 2 eq.) was added to the reaction mixture, followed by overnight stirring, under argon and at ambient temperature. Following the overnight reaction, the reaction was analyzed by thin layer chromatography (TLC; silica gel, 10:1 hexanes-ethyl acetate, visualization with UV). The TLC indicated a complete reaction, since the starting material was consumed and a new spot formed at a higher R_{f} from the starting material. The solid, produced during the overnight reaction, was filtered off through a bed of CELITE® and washed with 2×10 mL acetone. Next, the filtrate was concentrated to give very light yellow oil. This residue was purified by flash chromatography on a silica gel column (1×12 cm) using hexanes-ethyl acetate 30:1, 20:1 as eluents. This produced 112 mg (93%) of compound 2 as clear oil.

1-O-Carboxylmethyl-Δ⁹-THC (3).

The 1-O-EtOCOCH₂-Δ⁹-THC 2 (117 mg, 0.29 mmol) described above was dissolved in 5 mL of THF and 1 mL of methanol, followed by the addition of 5 mL 1N NaOH aqueous solution (FIG. 3). The reaction mixture was stirred for 4 hours, at room temperature (RT). The reaction was monitored by silica gel TLC (2:1 hexanes-ethyl acetate) to be sure the starting material was consumed. After completion, the reaction mixture was adjusted to pH=3 with 1N HCl and followed by extraction with ethyl acetate (30 ml×2). The extracted portions were washed with water (20 ml×2) and brine. Next, the combined organic phase was dried over anhydrous sodium sulfate. The drying was filtered off, and the solvent was evaporated. TLC check: 10:1 dichloromethane-methanol. The residue was purified by flash chromatography on a silica gel column (1×15 cm) using dichloromethane-methanol 25:1 and 20:1 as eluents. The product fractions were collected to give 59 mg (55%) of product 3 as sticky oil. TLC R_{f}0.50, dichloromethane-methanol 9:1.

1-O-(O-Succinimidyloxy)carbonylmethyl-Δ⁹-THC (4).

Next, 12.5 mg N-hydroxysuccinamide (NHS) (0.108 mmol, 1.5 equiv) and 20.8 mg 1-ethyl-3-(dimethylpropylamino)carbodiimide (EDC) hydrochloride (0.108 mmol, 1.5 equiv) were added to a solution of delta-9-THC-1-O—CH₂COOH 3 (27 mg, 0.0724 mmol, in 0.6 mL of anhydrous acetonitrile). This reaction mixture was stirred for 4 hours, at RT under a nitrogen atmosphere. The reaction mixture was used immediately in parallel for conjugation to three different proteins, KLH (70 uL), BGG (205 uL) and BSA (320 uL) as will be described in detail below.

Immunogen 5-KLH.

Referring to FIG. 3, 20 mg of KLH (0.01-0.00297 umol, 1 eq) was dissolved in 1 mL of deionized water. To this, 70 uL of the activated Δ⁹-THC-1-O—CH₂COOSu reaction mixture 4 (3.15 mg, 8.45 umol, 845-2845 equiv, see above) was added. Then 0.5 mL DMSO was added, to improve the solubility. The slightly cloudy reaction mixture was vortexed and stirred slowly overnight, at RT. The next day, the solution was directly loaded onto a pre-equilibrated G-50 SEPHADEX® column (1.5×25 cm). The column was eluted with 1× PBS buffer (pH 7.4, 0.05% sodium azide) at the flow rate of 1.2 ml/min. The eluent was monitored at 280 nm and the peak containing larger molecule /protein conjugated haptens (5-KLH) was collected. Dialysis of the reaction mixture with PBS buffer also provided the desired immunogen.

Immunogen 5-BGG.

To a solution of BGG (25 mg, 0.167 umol, 1 eq) in 1.2 mL of deionized water, 205 uL of the activated Δ⁹-THC-1-O—CH₂COOSu reaction mixture 4 (9.2 mg, 24.7 umol, 148 equiv) was added (see FIG. 3), followed by the addition of 1.0 mL DMSO. This cloudy reaction mixture was vortexed and slowly stirred overnight, at RT. The next morning, the reaction mixture was centrifuged and the supernatant decanted. The precipitate was then vortexed with 1 mL PBS buffer, centrifuged, and the supernatant loaded onto a pre-equilibrated G-50 SEPHADEX®column (1.5×25 cm).The column was eluted with 1× PBS (pH 7.4, 0.05% sodium azide) at the flow rate of 1.2 ml/min. The eluent was monitored at 280 nm and the peak containing larger protein conjugated haptens (5-BGG) collected. Dialysis of the reaction mixture with PBS buffer also provided the desired immunogen.

Immunogen 5-BSA.

320 uL activated Δ⁹-THC-1-O—CH₂COOSu reaction mixture 4 (14.4 mg 38.6 umol, 105 equiv) was added to a solution of BSA (25 mg, 0.368 umol, 1 eq) in 1.2 mL of deionized water (FIG. 3). Next, 1.5 mL of DMSO was added to the solution. The resulting cloudy reaction mixture was vortexed and slowly stirred overnight, at RT. The next day, the reaction mixture was centrifuged and the supernatant decanted. The precipitate was then dissolved in I mL PBS and centrifuged again. The resulting supernatant was loaded on a pre-equilibrated G-50 SEPHADEX® column (1.5×25 cm). The column was eluted with 1× PBS (pH 7.4, 0.05% sodium azide) at a flow rate of 1.2 ml/min. The eluent was monitored at 280 nm and the peak containing larger protein conjugated haptens (5-BSA) collected. Dialysis of the reaction mixture with PBS buffer also provided the desired immunogen.

Activation of 3 and Conjugation With KLH

An alternative activation of 3 and conjugation onto KLH for the synthesis of immunogen 5-KLH can also be performed. Compound 3 was activated and conjugated onto KLH by the following protocol: To a solution of delta-9-THC-1-O—CH₂COOH 3 (15 mg, 0.04 mmol) in 0.5 mL of anhydrous acetonitrile were added N-hydroxysuccinamide (NHS) (6.96 mg, 0.06 mmol, 1.5 equiv) and 1-ethyl-3-(dimethylpropylamino)carbodiimide (EDC) hydrochloride (11.5 mg, 0.06 mmol, 1.5 equiv). The reaction mixture was stirred at room temperature under a nitrogen atmosphere for 4 hours.

Immunogen 5-KLH

KLH (60 mg, 0.03-0.0089 umol, 1 eq) was dissolved in 3.5 mL of 50% diluted 1× PBS buffer. The activated delta-9-THC-1-O—CH₂COOSu reaction mixture 4 (1300-4400 equiv) obtained above was added to the KLH solution. The slightly cloudy reaction mixture was stirred slowly at room temperature overnight. The reaction mixture was transferred to a dialysis bag and dialyzed with 5% DMSO in 1× PBS buffer for one day, and then dialyzed 5 days with 1× PBS buffer containing 0.05% sodium azide. The buffer was changed once each day. The trace amount of precipitate was centrifuged out, and 5.4 mL of clear pale blue immunogen solution 5-KLH was obtained with the concentration of 20.9 mg/mL measured by the Coomassie assay.

### EXAMPLE 2

### Synthesis of Dihydroxy-Δ⁹-THC Antigens

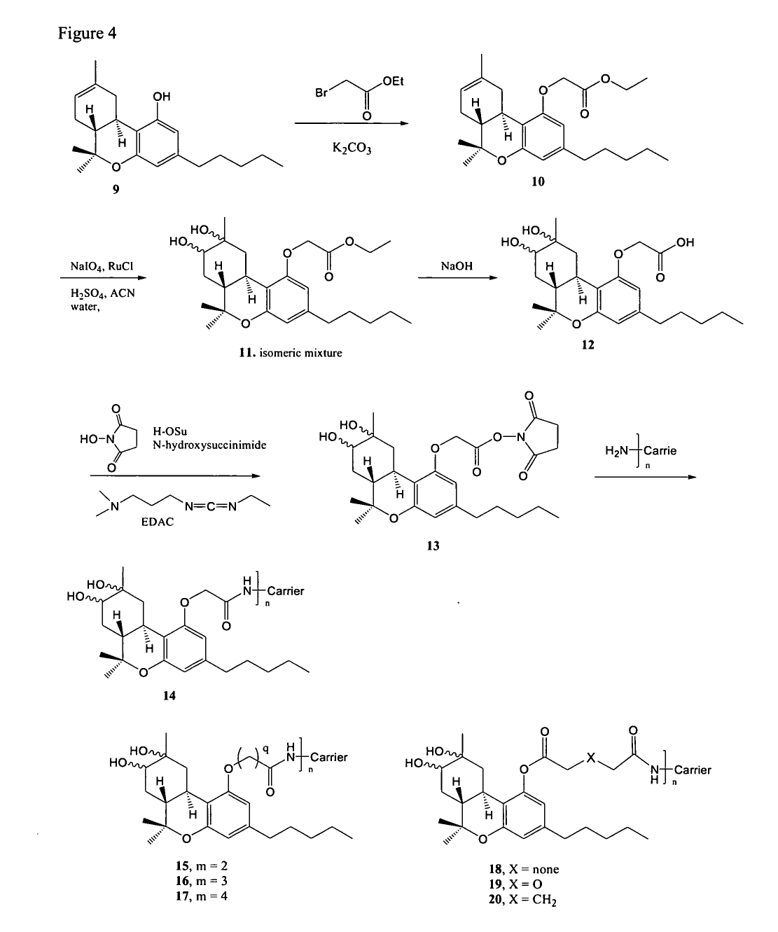

Tetrahydrocannabinol derivatives are very lipophilic (see FIG. 4). In order to increase the water solubility of the immunogens, di-hydroxyl substituted THC was designed and synthesized as a hapten. Δ⁸-derivative 10 was synthesized as described herein from Δ⁸-THC. Next, the double bond of the Δ⁸-THC was oxidized and hydrolyzed to give the corresponding hydroxyl compound 11 as an isomeric mixture. Compound 11 was hydrolyzed, activated and then conjugated onto the carriers, resulting in the desired immunogens. The new immunogens were purified by the same means as described above. Immunogens 15-17 were synthesized by a similar strategy from different halo-esters. Immunogens 18-20 were synthesized by a similar strategy as illustrated in FIG. 4. The novel immunogens 14-20, without a double bond in the hapten structures, are used to elicit specific antibodies for Δ⁹- and Δ⁸-THC. The antibodies may also recognize metabolites of THC.

1-O-Ethyloxycarbonylmethyl-Δ⁸-THC (10).

Referring to FIG. 4, a methanol solution of 8A-THC (9) (300 mg, 0.954 mmol, 30 vials of 10 mg/vial) was transferred to a round bottom flask. The original vials were washed twice with methanol, and the washes added to the flask. Third washes of the vials were kept for TLC analysis. Next, the solvent was removed using a rotary evaporator, producing a light brown oil. This light brown oil, the isolated ⁸A-THC, was dissolved in 30 mL acetone (dried over K₂CO₃) followed by the addition of 1.32 g potassium carbonate (9.54 mmol). Ethyl bromoacetate was added to the reaction mixture (212 uL, 319 mg, 1.91 mmol, 2 eq.) followed by overnight stirring, under argon at ambient temperature.

The next day, the reaction was monitored by TLC (silica gel, 10:1 hexanes-ethyl acetate, visualization with UV, I₂). The TLC indicated a complete reaction because the starting material was consumed and a new spot formed at a higher R_{f} from the starting material. Next, the solid was filtered off through a bed of CELITE®. Then the solid was then washed with acetone (3×20 mL) and the filtrate concentrated to give very light yellow oil. This residue was purified by flash chromatography on a silica gel column (3×15 cm) using hexanes-ethyl acetate 30:1 and 20:1 as eluents. This produced 327 mg (85.6%) of the desired product 10 as a pale yellow oil. Silica gel TLC R_{f}=0.58 (dichloromethane-methanol 10:1).

8,9-Dihydroxy Compound 11.

Compound 11 was synthesized in the following manner (see FIG. 4). 327 mg of⁸Δ-THC ethyl ester 10 (0.816 mmol) was dissolved in a mixture of ethyl acetate (6 mL) and acetonitrile (6 mL), and then cooled to 0° C. To the chilled solution, a solution of Ruthenium (III) chloride hydrate (RuCl₃ 3H₂O) (36.5 mg, 0.14 mmol, 0.17 eq. and sodium (meta) periodate (NaIO₄) (262 mg, 1.22 mmol, 1.5 eq.) in 2 mL of distilled water was added. The two phase reaction mixture was stirred vigorously for 10 min (TLC, hexanes-ethyl acetate 10:1 to check the disappearance of starting material) (total 15 minutes). Next, 20 mL of a saturated sodium thiosulfate (Na₂S₂O₃) solution was added to quench the reaction mixture, followed by the addition of 20 mL ethyl acetate. The resulting phases, a black organic phase and an aqueous phase, were separated. The black organic phase was washed twice with brine while the aqueous phase was extracted with ethyl acetate (3×20 mL). The ethyl acetate extractions were combined with the black organic phase, and then the combined organic phase was dried and concentrated. This produced a black residue, which was purified by flash chromatography on a silica gel column (3×16 cm) using 3:1, 2:1 and 1:2 hexanes-ethyl acetate as eluents. 245.7 mg (69.4%) of product 11 was produced as a pale yellow oil.

8,9-Dihydroxy Carboxylic Acid Compound 12.

Still referring to FIG. 4, 240 mg of 1-O-ethxoycarbonyl-8,9-dihydroxy-THC 11 (0.55 mmol) was dissolved in 3 mL of THF and 4 mL of methanol. Next, 10 mL of aqueous 1N NaOH was added. The reaction mixture was stirred at RT for 4 hours and the reaction was monitored by silica gel TLC (1:1 hexanes-ethyl acetate). At completion of the reaction, the reaction mixture was adjusted to pH=3 with 1N HCl. Next, the mixture was extracted with ethyl acetate (30 ml×3). The extracted portions were combined, washed with water (20 ml×2) and brine, and then dried over anhydrous sodium sulfate. The drying was filtered off, and the solvent evaporated to give 204 mg (91%) product 12 as white foam. TLC R_{f}=0.46 and 0.43 dichloromethane-methanol 9:1 (the two close spots represent the isomers on the hydroxyl carbon chiral centers).

Activated Ester Compound 13.

Still referring to FIG. 4, 20.7 mg of N-hydroxysuccinamide (NHS, 0.18 mmol, 1.5 equiv) and 34.5 mg of 1-ethyl-3-(dimethylpropylamino)carbodiimide (EDC) hydrochloride (0.18 mmol, 1.5 equiv) were added to a solution of 8,9-di-OH-delta-8-THC-1-O—CH₂COOH 12 (48.8 mg, 0.12 mmol, in 1.0 mL of anhydrous acetonitrile). The reaction mixture was stirred under a nitrogen atmosphere for 4 hours, at RT. The reaction mixture was then used immediately, in parallel, for conjugation to 3 separate proteins: KLH (140 uL), BGG (340 uL) and BSA (510 uL) (see below).

Immunogen 14-KLH.

To conjugate compound 14 to KLH, 40 mg of KLH (0.02-0.00594 umol, 1 eq) was dissolved in 2 mL deionized water. Nest, 140 uL of the activated di-OH-delta-8-THC-1-O—CH₂COOSu reaction mixture 13 (6.8 mg, 16.8 umol, 840-2828 equiv) was added to the KLH-water solution. 1.0 mL of DMSO was then added, to improve the solubility. This slightly cloudy reaction mixture was vortexed and slowly stirred overnight at RT and then loaded onto a pre-equilibrated G-50 SEPHADEX® column (1.5×24 cm). The column was eluted with 1× PBS (pH 7.4, 0.05% sodium azide) at a flow rate of 1.2 ml/min. The eluent was monitored at 280 nm and the peak containing the larger protein conjugated haptens (14-KLH) collected.

Immunogen 14-BGG.

Compound 14 was also conjugated to BGG by the following procedure (see FIG. 4). A solution of BGG (40 mg, 0.266 umol, 1 eq) in deionized water (2.0 mL) was added to 340 uL of activated di-OH-delta-8-THC-1-O—CH₂COOSu reaction mixture 13 (16.6 mg, 40.8 umol, 153 equiv). Next, 2.0 mL DMSO was added. The resulting cloudy reaction mixture was vortexed and slowly stirred overnight, at RT. The next day, the reaction mixture was centrifuged and the supernatant decanted. The precipitate was then vortexed in 1 mL PBS and centrifuged. The resulting supernatant was loaded onto a pre-equilibrated G-50 SEPHADEX® column (1.5×24 cm). The column was eluted with 1× PBS (pH 7.4, 0.05% sodium azide) at a flow rate of 1.2 ml/min. The eluent was monitored at 280 nm. The peak containing the larger protein conjugated haptens (14-BGG) was collected.

Immunogen 14-BSA.

Referring to FIG. 4, compound 14 was also conjugated to BSA. Activated di-OH-delta-8-THC-1-O—CH₂COOSu reaction mixture 13 (510 uL, 24.89 mg 61.2 umol, 104 equiv) was added to a solution of BSA (40 mg, 0.588 umol, 1 eq) in 2.0 mL deionized water. 3.0 mL of DMSO was then added to produce a cloudy reaction mixture. This mixture was vortexed and slowly stirred overnight, at RT. Then reaction mixture was centrifuged, and the precipitate was vortexed in 1 mL of PBS buffer. This was followed by centrifugation, and the supernatant was loaded onto a pre-equilibrated G-50 Sephadex column (1.5×24 cm). After this, the column was eluted with 1× PBS (pH 7.4, 0.05% sodium azide) at a flow rate of 1.2 ml/min. The eluent was monitored at 280 nm. The peak containing larger molecule /protein conjugated haptens (14-BSA) was collected.

Activated Ester Compound 13

Referring to FIG. 4, compound 12 was alternatively activated and conjugated onto KLH by the following protocol: To a solution of8,9-di-OH-delta-8-THC-1-O—CH₂COOH 12 (95 mg, 0.233 mmol) in 2.0 mL of anhydrous acetonitrile were added N-hydroxysuccinamide (NHS) (32.7 mg, 0.28 mmol, 1.2 equiv) and 1-ethyl-3-(dimethylpropylamino)carbodiimide (EDC) hydrochloride (53.7 mg, 0.28 mmol, 1.2 equiv). The reaction mixture was stirred at room temperature under a nitrogen atmosphere for 4 hours.

Immunogen 14-KLH

KLH (60 mg, 0.03-0.0089 umol, 1 eq) was dissolved in 3.5 mL of 50% diluted 1× PBS buffer. The activated di-OH-delta-8-THC-1-O—CH₂COOSu reaction mixture 13 (230 uL, 10.9 mg, 26.8 umol, 890-3000 equiv) was added to the KLH solution. The slightly cloudy reaction mixture was stirred slowly at room temperature overnight. The reaction mixture was transferred to a dialysis bag and dialyzed with 5% DMSO in 1× PBS buffer for one day, and then dialyzed 5 days with 1× PBS buffer containing 0.05% sodium azide. The buffer was changed once each day. The trace amount of precipitate was centrifuged out, and 4.5 mL of clear immunogen solution 14-KLH was obtained with the concentration of 22.5 mg/mL measured by Coomassie assay.

### EXAMPLE 3

### Synthesis of Δ⁹-THC-CH₂═CHCO Antigens

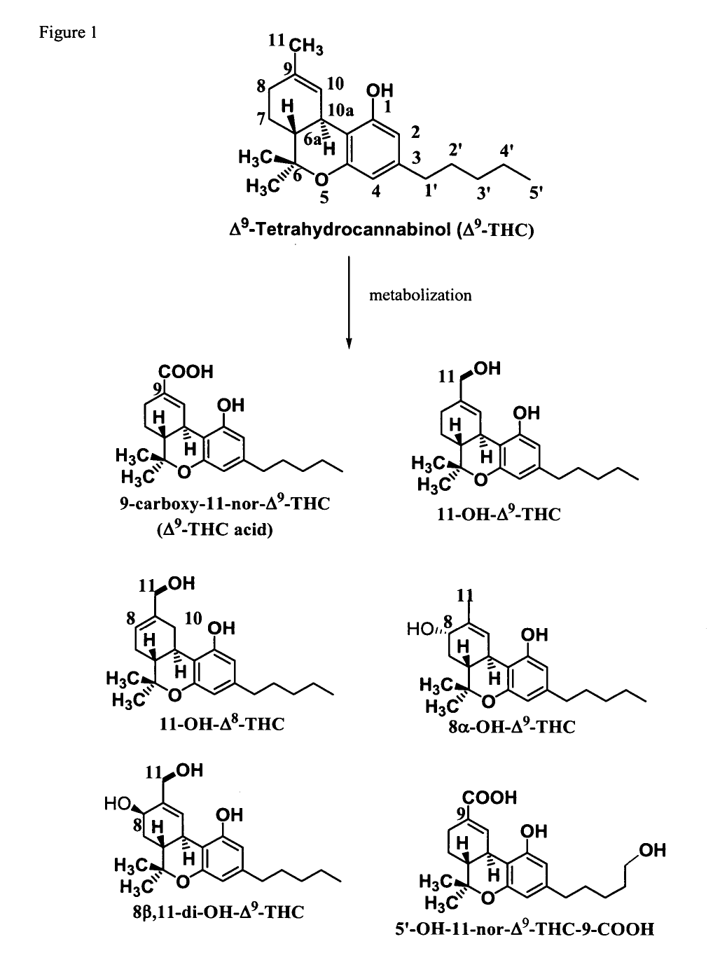

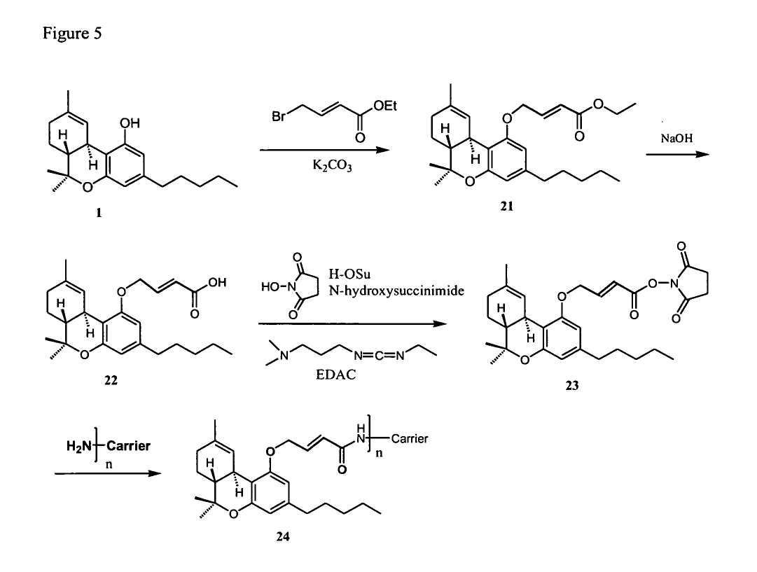

Referring to FIG. 5, immunogen 24 was designed and synthesized to test the effect of the different spacers. The spacer of 24 is one double bond longer than that of immunogen 5 (FIG. 1). Immunogen 24 was synthesized by the same methods described above (for the synthesis of immunogen 5) starting from ethyl 4-(bromo)crotonate instead of a saturated halo-ester.

Ethyl Δ⁹-THC-1-O-crotonate (21).

An ethanol solution of ⁹Δ-THC (1) (100 mg, 0.318 mmol) was transferred to a round bottom flask (FIG. 5). The original vial was washed with methanol and the washes added to the flask. Next the solvent was removed on the rotary evaporator, to give yellow oil. The isolated ⁹Δ-THC yellow oil was dissolved in 10 mL acetone (dried over K₂CO₃) followed by the addition of 0.45 g potassium carbonate (3.2 mmol). Ethyl 4-bromocrotomate (118 uL, 165 mg×75%=124, 0.64 mmol, 2 eq.) was then added to the mixture. This RT reaction was stirred overnight, under argon. In the morning, the reaction was analyzed by TLC (silica gel, 10:1 hexanes-ethyl acetate, visualization with UV). The TLC indicated a complete reaction, the starting material having been consumed and a new spot having formed at a higher R_{f} from the starting material. Next, the solid was filtered off through a bed of CELITE®. The solid was then washed with acetone (2×10 mL). The filtrate was concentrated to give very light yellow oil. This residue was then purified by preparative TLC on a silica gel using hexanes-ethyl acetate 10:1 as eluent. The top band (R_{f}=0.57) was collected to give 135 mg (99%) of the desired product 21 as a clear oil.

Δ⁹-THC-1-O-crotonic Acid Compound 22.

Referring to FIG. 5, 124 mg of 1-O-EtOCOCH═CHCH₂-delta-9-THC 21 (0.29 mmol) was dissolved in 6 mL of a THF/methanol solution (1:1), followed by the addition of 5 mL aqueous 1N NaOH. The reaction mixture was stirred 4 hours, at RT. The reaction was monitored by silica gel TLC (10:1 hexanes-ethyl acetate) to be sure the starting material disappeared. At completion of the reaction, the reaction mixture was adjusted to pH =3 with 1N HCl, followed by extraction with ethyl acetate (30 ml×2). The extracted portions were washed with water (20 ml×2) and brine. The combined organic phase was dried over anhydrous sodium sulfate. The drying was filtered off, and the solvent evaporated (TLC check: 20:1 dichloromethane-methanol). The resulting residue was purified by flash chromatography on a silica gel column (1×15 cm) using dichloromethane-methanol 50:1 and 40:1 as eluents. The product fractions were collected to give 80.9 mg (70%) product 22 as white foam. TLC R_{f}=0.48 and 0.41, dichloromethane-methanol 20:1 (the two close spots represent cis/trans isomers of the double bond).

Activated Ester Compound 23.

Next, to a solution of 29 mg Δ⁹-THC-1-O—CH₂CH═CHCOOH 22 (0.0727 mmol) in 0.60 mL anhydrous acetonitrile were added 12.55 mg N-hydroxysuccinamide (NHS, 0.109 mmol, 1.5 equiv) and 20.9 mg 1-ethyl-3-(dimethylpropylamino)carbodiimide (EDC) hydrochloride (0.109 mmol, 1.5 equiv)(see FIG. 5). This reaction mixture was stirred at RT under a nitrogen atmosphere for 4 hours. The reaction mixture was then used immediately in parallel for conjugation to 3 proteins: KLH (70 uL), BGG (205 uL) and BSA (320 uL).

Immunogen 24-KLH.

Referring to FIG. 5, 20 mg KLH (0.01-0.00297 umol, 1 eq) was dissolved in 1 mL deionized water. Next, 70 uL of the activated delta-9-THC-1-O—CH₂CH═CHCOOSu reaction mixture 23 (3.38 mg, 8.49 umol, 849-2858 equiv) was added to the dissolved KLH. Then 0.5 mL of DMSO was added. This slightly cloudy reaction mixture was vortexed and slowly stirred overnight, at RT. The next day, the reaction mixture was centrifuged and the supernatant decanted. The precipitate was vortexed in 1 mL of PBS buffer and centrifuged. The resulting supernatant was loaded onto a pre-equilibrated G-50 medium SEPHADEX® column (1.5×20 cm). The column was eluted with 1× PBS (pH 7.4, 0.05% sodium azide) at a flow rate of 1.2 ml/min. The eluent was monitored at 280 nm. The peak containing larger protein conjugated haptens (24-KLH) was collected.

Immunogen 24-BGG.

The activated delta-9-THC-1-O—CH₂CH═CHCOOSu reaction mixture 23 (205 uL, 9.9 mg, 24.86 umol, 148 equiv) was added to a solution of BGG (25 mg, 0.167 umol, 1 eq) in 1.2 mL deionized water (FIG. 5). 1.0 mL of DMSO was then added to improve the solubility. The cloudy reaction mixture was vortexed and stirred slowly overnight, at RT. The next day, the reaction mixture was centrifuged and the supernatant decanted. The precipitate was vortexed in 1 mL of PBS buffer, centrifuged, and the supernatant loaded onto a pre-equilibrated G-50 fine SEPHADEX® column (1.5×20 cm). The column was eluted with 1× PBS (pH 7.4, 0.05% sodium azide) at a flow rate of 1.2 ml/min. The eluent was monitored at 280 nm. The peak containing larger protein conjugated haptens (24-BGG) was collected.

Immunogen 24-BSA.

Still referring to FIG. 5, a solution of 25 mg BSA (0.368 umol, 1 eq) in 1.2 mL of deionized water was added to 320 uL of the activated delta-9-THC-1-O—CH₂CH═CHCOOSu reaction mixture 23 (15.46 mg, 38.8 umol, 105 equiv). 1.5 mL of DMSO was then added. The cloudy reaction mixture was vortexed and slowly stirred overnight, at RT. The reaction mixture was centrifuged and the supernatant decanted. The precipitate was vortexed with 1 mL of PBS, centrifuged, and then the supernatant was loaded onto a pre-equilibrated G-50 fine SEPHADEX® column (1.5×20 cm). The column was eluted with 1× PBS (pH 7.4, 0.05% sodium azide) at a flow rate of 1.2 ml/min. The eluent was monitored at 280 nm. The peak containing larger protein conjugated haptens (24-BSA) was collected. Immunogens 24-KLH, 24-BGG, and 24-BSA were also purified by dialysis with PBS buffer.

### EXAMPLE 4

### Synthesis of Δ⁹-THC-Ester Antigens

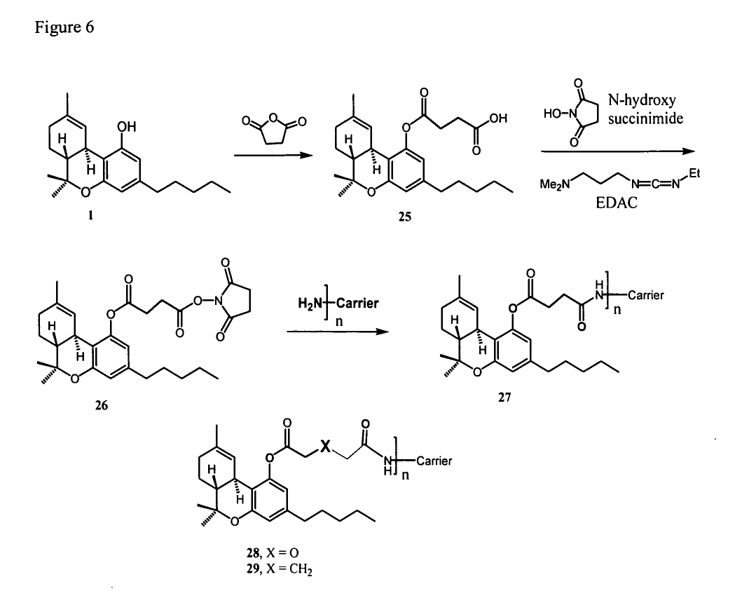

Referring to FIG. 6, ⁹Δ-tetrahydrocannabinol (1) was reacted with succinic anhydride to give the hemisuccinic ester 25 of ⁹A-THC. The carboxylic acid group of 25 was activated and conjugated onto the carrier proteins as described above. Antigens 28 and 29 represent the different types of spacers having an ester connection with the haptens although the amide conjugation is still the same as before for other immunogens.

Δ⁹-THC Hemisuccinate Ester 25.

The ethanol solution of ⁹Δ-THC (1) (100 mg, 0.318 mmol) was transferred to a round bottom flask (see FIG. 6). The original vial was washed with methanol, the washes added to the flask and the solvent removed on the rotary evaporator, to give yellow oil. The isolated ⁹Δ-THC was dissolved in 10 mL pyridine followed by the addition of 320 mg succinic anhydride (3.19 mmol, 10 eq). The reaction was stirred under argon at ambient temperature followed by overnight heating (70° C.), and then concentrated, and co-evaporated with toluene. The resulting residue was dissolved in ethyl acetate, and washed with water (×3) and brine. The ethyl acetate solution was concentrated, and the resulting residue purified by chromatography on a silica gel column using dichloromethane-methanol 50:1, 40:1 and 20:1. The major fractions were collected to give pale brown oil (170 mg). The pale brown oil was further purified by preparative TLC (DCM-MeOH 10:1). The top band was collected to give 79 mg (60%) of the desired product 25, as pale yellow foam.

Activated Ester Compound 26.

To a solution of delta-9-THC-1-O—COCH₂CH₂COOH 25 (39 mg, 0.094 mmol) in anhydrous acetonitrile (0.60 mL) were added 16.1 mg N-hydroxysuccinamide (NHS; 0.14 mmol, 1.5 equiv) and 26.8 mg 1-ethyl-3-(dimethylpropylamino)carbodiimide (EDC) hydrochloride (0.14 mmol, 1.5 equiv). The reaction mixture was stirred at RT under a nitrogen atmosphere for 4 hours. The reaction mixture was then used immediately, in parallel, for conjugation to three proteins: KLH (70 uL), BGG (205 uL) and BSA (320 uL) (see FIG. 6).

Immunogen 27-KLH.

One vial of KLH (20 mg, 0.01-0.00297 umol, 1 eq) was dissolved in 1 mL of deionized water. Next, 70 uL activated Δ⁹-THC-1-O—COCH₂CH₂COOSu reaction mixture 26 (4.55 mg, 10.9 umol, 1100-3700 equiv) was added. 0.5 mL of DMSO was subsequently added. The resulting slightly cloudy reaction mixture was vortexed and slowly stirred overnight, at RT. The next morning, the reaction mixture was centrifuged and the supernatant decanted. The precipitate was then vortexed with 1 mL of PBS buffer and centrifuged. The resulting supernatant was loaded onto a pre-equilibrated G-50 medium SEPHADEX(D column (1.5×20 cm). After loading, the column was eluted with 1× PBS buffer (pH 7.4, 0.05% sodium azide) at a flow rate of 1.2 ml/min. The eluent was monitored at 280 nm. The peak containing conjugated hapten (27-KLH) was collected (see FIG. 6).

Immunogen 27-BGG.

To a solution of BGG (20 mg, 0.133 umol, 1 eq in 1.2 mL deionized water) was added 205 uL of the activated Δ⁹-THC-1-O—COCH₂CH₂COOSu reaction mixture 26 (13.3 mg, 32 umol, 240 equiv). 1.0 mL of DMSO was then added to improve the solubility. The resulting cloudy reaction mixture was vortexed and slowly stirred overnight, at RT. The next day, the reaction mixture was centrifuged and the supernatant poured off. The precipitate was dissolved in 1 mL of PBS buffer, centrifuged, and the supernatant loaded on a pre-equilibrated G-50 fine SEPHADEX® column (1.5×20 cm). The column was eluted with 1× PBS buffer (pH 7.4, 0.05% sodium azide) at a flow rate of 1.2 ml/min. The eluent was monitored at 280 nm. The peak containing conjugated hapten (27-BGG) was collected (see FIG. 6).

Immunogen 27-BSA.

To a solution of BSA (20 mg, 0.294 umol, 1 eq in 1.2 mL deionized water), the activated Δ⁹-THC-1-O—COCH₂CH₂COOSu reaction mixture 26 (320 uL, 20.8 mg, 50 umol, 170 equiv) was added. Then 1.0 mL of DMSO was added. The resulting cloudy reaction mixture was vortexed and slowly stirred overnight, at RT. The next morning, the reaction mixture was centrifuged and the supernatant discarded. The precipitate was vortexed with 1 mL of PBS buffer, centrifuged, and the supernatant loaded on a pre-equilibrated G-50 fine SEPHADEX® column (1.5×20 cm). The column was eluted with 1× PBS buffer (pH 7.4, 0.05% sodium azide) at a flow rate of 1.2 ml/min. The eluent was monitored at 280 nm. The peak containing conjugated hapten (27-BSA) was collected (see FIG. 6). The immunogens 27-KLH, 27-BGG, 27-BSA, 28, and 29, were also purified by dialysis with PBS buffer.

### EXAMPLE 5

### Synthesis of Δ⁹-THC-CH₂C₆H₄CO Antigens

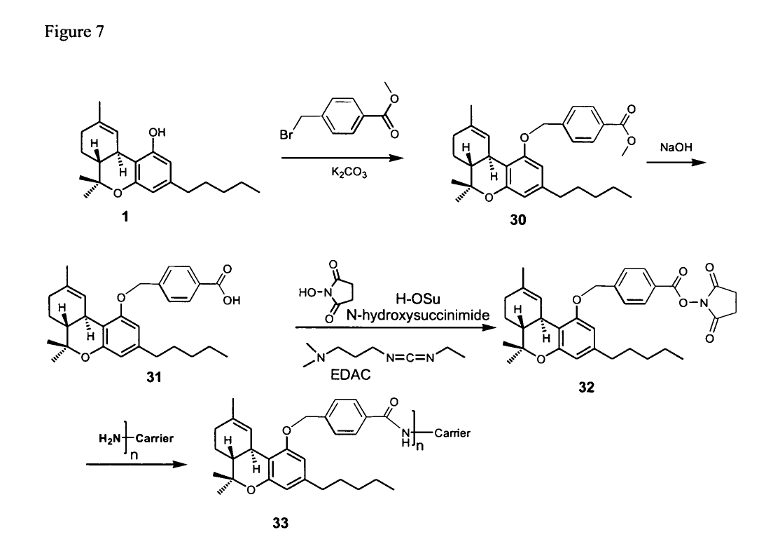

Immunogens 33 (FIG. 7) were designed and synthesized to test the effect of the different spacers. The spacer of 33 is longer than those of immunogens 5-8 (FIG. 3), with higher rigidity. Immunogens 33 were synthesized by the same method described above for the synthesis of immunogen 5, except starting with methyl 4-(bromomethyl) benzoate instead of ethyl bromoacetate.

1-O-(4-Methoxycarbonyl)benzyl-Δ⁹-tetrahydrocannabinol (30).

An ethanol solution of ⁹Δ-tetrahydrocannabinol (1) (100 mg, 0.32 mmol) was transferred to a round bottom flask (FIG. 7). The original vial was washed with methanol and the methanol wash was added to the flask. The solvent was then removed on a rotary evaporator to produce a yellow oil. The ⁹Δ-THC (1) yellow oil was dissolved in 10 mL of acetone (dried over K₂CO₃) followed by the addition of 110 mg anhydrous potassium carbonate (0.80 mmol). To the reaction mixture 146 mg methyl 4-(bromomethyl) benzoate (0.64 mmol, 2 eq) was added. This reaction mixture was stirred overnight under argon, at RT. The reaction was analyzed by TLC (silica gel, 10:1 hexanes-ethyl acetate, visualization with UV). The TLC indicated a complete reaction, showing a spot with a higher R_{f} value than the starting material. The reaction mixture was filtered through a bed of Celite. The Celite bed was washed with acetone (2×10 mL) and the filtrate concentrated to give yellow oil. This residue was passed through a pad of silica gel. The major fractions containing product were combined and their solvent was evaporated. The resulting residue 30 was taken to the next step without further purification.

Δ⁹-Tetrahydrocannabinol-1-O-methyl 4-benzoic Acid Compound 31.

1-O-(4-Methoxycarbonyl)benzyl-Δ⁹-tetrahydrocannabinol 30 (147 mg, 0.32 mmol) was dissolved with 3 mL of THF plus 3 mL methanol (FIG. 7). 5 mL of aqueous 1 N NaOH were added to the solution. The reaction mixture was stirred overnight at RT. The reaction was monitored by silica gel TLC (10:1 hexanes-ethyl acetate). The solvents were evaporated off, and the resulting residue purified by flash chromatography on a silica gel column using dichloromethane and methanol (20:1 through 5:1) as eluents. This was followed by preparative TLC purification using a dichloromethane and methanol (8:1) solvent system. The desired product 31 (19 mg, 13% over two reactions) was obtained as a white foam. TLC R_{f}=0.45 dichloromethane-methanol 10:1.

Activated Ester Compound 32.

Referring to FIG. 7, to a solution of Δ⁹-Tetrahydrocannabinol-1-O-methyl 4-benzoic Acid 31 (18 mg, 0.04 mmol in 0.50 mL of anhydrous acetonitrile) were added 7.0 mg N-hydroxysuccinamide (NHS; 0.06 mmol, 1.5 eq) and 12 mg 1-ethyl-3-(dimethylpropylamino)carbodiimide (EDC) hydrochloride (0.06 mmol, 1.5 eq). The reaction mixture was stirred overnight at RT, under a nitrogen atmosphere. The next morning, the reaction mixture was diluted with 1.5 mL of DMSO. The resulting solution 32 was used immediately for conjugation to 2 proteins in a parallel: KLH (557 EL), and BGG (1447 μL), as described below.

Immunogen 33-KLH.

One vial of KLH (20 mg, 0.01-0.00297 umol, 1 eq) was dissolved in 1.0 mL deionized water (see FIG. 7). 557 μL of the activated ester 32 reaction mixture (5 mg, 11 μmol, 849-2858 eq) was added to the KLH-water solution. The slightly cloudy reaction mixture was vortexed and slowly stirred overnight, at RT. The next morning, the reaction mixture was centrifuged, and the supernatant discarded. Thens, the precipitate was vortexed in 1 mL PBS, centrifuged, and the supernatant was loaded onto a pre-equilibrated G-50 medium SEPHADEX® column (1.5×20 cm). The column was eluted with 1× PBS buffer (pH 7.4, 0.05% sodium azide) at a flow rate of 1.2 ml/min. The eluent was monitored at 280 nm. The peak containing larger molecule (protein with conjugated haptens, 33-KLH) was collected.

Immunogen 33-BGG.

To a solution of BGG (20 mg, 0.133 μmol, 1 eq, in 1.0 mL deionized H₂O) was added 1447 μL of the activated ester 32 reaction mixture (13 mg, 29 μmol, 217 eq). The cloudy reaction mixture was vortexed and slowly stirred overnight, at RT. The reaction mixture was centrifuged, and the supernatant discarded. The precipitate was vortexed in 1 mL of PBS buffer, centrifuged, and the supernatant loaded onto a pre-equilibrated G-50 fine SEPHADEX® column (1.5×20 cm). The column was eluted with 1× PBS buffer (pH 7.4, 0.05% sodium azide) at a flow rate of 1.2 ml/min. The eluent was monitored at 280 nm. The peak containing larger molecule (protein with conjugated haptens, 33-BGG) was collected. These immunogens were also purified by dialysis with PBS buffer.

### EXAMPLE 6

### Synthesis of Optically Pure 11-nor-Δ⁹-THC-9-Carboxylic Acid and Related Antigens

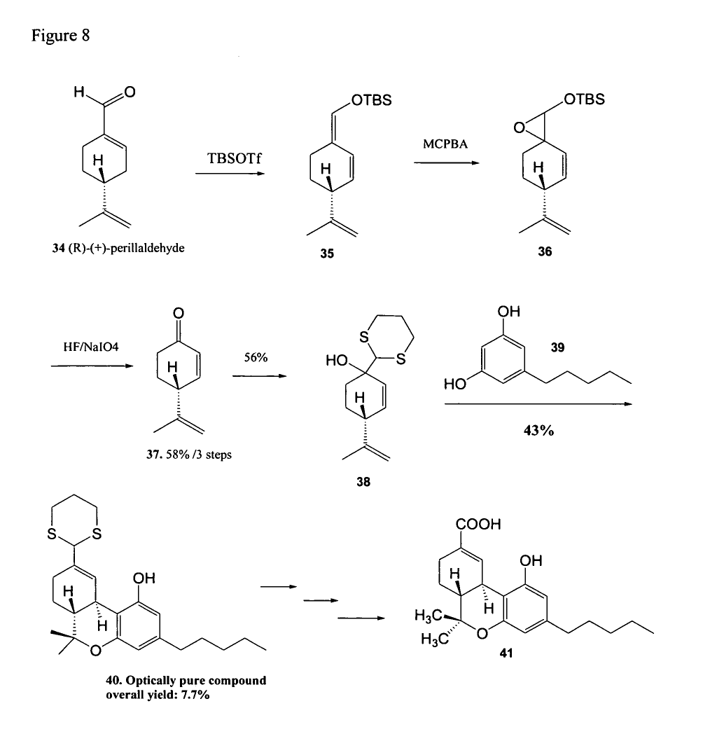

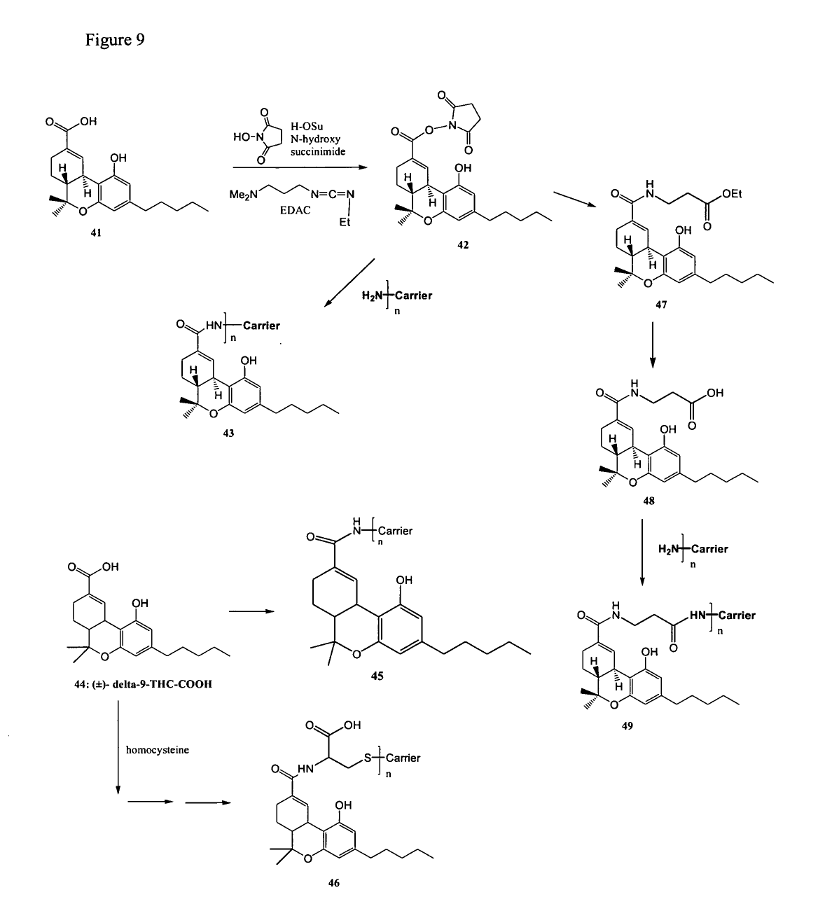

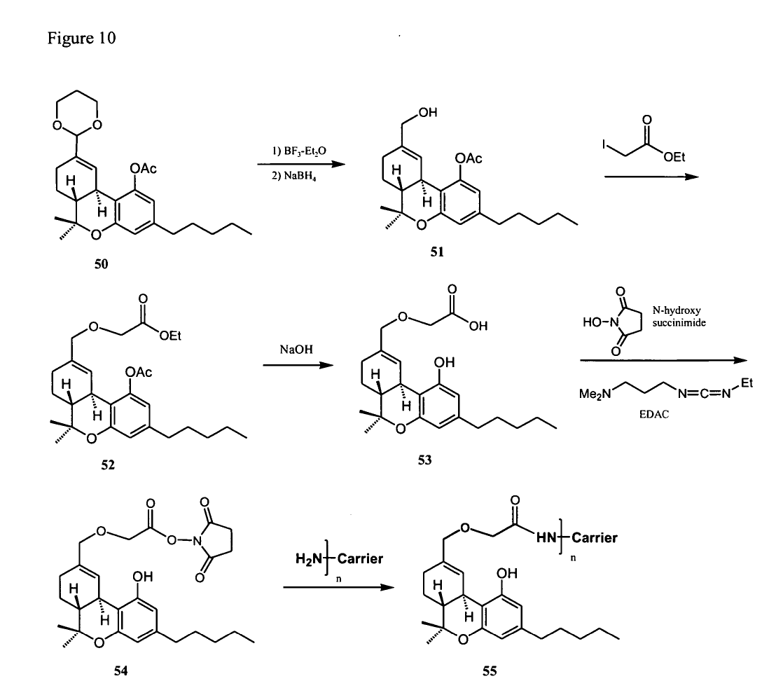

Referring to FIGS. 8-10, in order to conjugate the THC haptenic derivatives onto proteins through position 9, the key intermediates and haptens 41, 48, 51, and 53 were synthesized. The synthesis of optically pure 11-nor-Δ⁹-tetrahydrocannabinol-9-carboxylic acid 41 was done according to the reported procedures with modifications (Ting Liang, et al. Biosci. Biotechnol. Biochem. 66, 2501-2503 (2002); Craig Siegel, et al. J. Org. Chem. 54, 5428-5430 (1989); Craig Siegel, et al. J. Org. Chem. 56, 6865-6872 (1991); Seung-Hwa Baek, et al. Pharmcol. Biochem. Behavior 40, 487-489 (1991)). The synthesis is depicted in FIG. 8. The racemic mixture 44 was synthesized according to the reported procedures (Huffman, J. W. et al., J. Org. Chem., 54, 4741-54 (1989); Uliss, D. B. et al. J. Am. Chem. Soc. 100, 2929-30 (1978). Hapten 41 was activated and conjugated onto carrier proteins to provide immunogen 43. Similarly, hapten 44 was activated and conjugated onto a carrier to provide immunogen 45. Compound 44 was also reacted with acety protected homocysteine. The resulting product was further conjugated onto BSA to give immunogen 46-BSA. Hapten 41 was converted to 48, which was then also conjugated onto the carrier proteins through longer spacers to give immunogens 49 (FIG. 9). The key intermediate 50 was modified to the 1 1-hydroxy-Δ⁹-THC derivative 51, which was then conjugated onto the carrier proteins to result in immunogen 55 (FIG. 10).

Activation of 11-nor-delta-9-THC-9-COOH 41: To a solution of 11-nor-delta-9-THC-9-COOH 41 (3.0 mg, 8.7 umol) in 0.2 mL of anhydrous acetonitrile were added N-hydroxysuccinamide (NHS) (1.50 mg, 13 umol, 1.5 eq) and 1-ethyl-3-(dimethylpropylamino)carbodiimide (EDC) hydrochloride (2.5 mg, 13 umol, 1.5 eq). The reaction mixture was stirred at room temperature for 4 hours, and then used immediately for next step.

Immunogen 43-KLH: KLH (20 mg, 0.01-0.00297 umol) was dissolved in 2 mL of 1× PBS buffer (diluted into 50%) and cooled to 5° C. The reaction mixture obtained above containing activated ester 42 was added slowly. The reaction mixture was stirred slowly at room temperature overnight. The reaction mixture was dialyzed with 5% DMSO in 1× PBS buffer for one day, and then 1× PBS buffer containing 0.05% sodium azide for 5 days. The buffer was changed once each day. The trace amount of precipitate was centrifuged out. 3.2 mL resulting immunogen solution in 1× buffer with 0.05% azide was tested with Commassie assay to show 10.55 mg/mL concentration.

### EXAMPLE 7

### Synthesis of Immunogens from Racemic Mixture of 11-nor-delta-9-THC-9-Carboxylic Acid

Immunogen 45-BSA: A racemic mixture of 11-nor-delta-9-THC-COOH (±)-44 activated by N-hydroxysuccinamide and EDC (as described above) was conjugated onto carrier BSA to provide the immunogen 45-BSA.

Immunogen 46-BSA: To a solution of (±)-11-nor-Δ⁹-THC-COOH 44 (98 mg, 0.285 mmol) in 3 mL of anhydrous pyridine was added DL-homocysteine thiolactone hydrochloride (50 mg, 0.325 mmol) and EDCI (108 mg, 0.563 mmol). The mixture was stirred at room temperature for 6 hrs under nitrogen. The solvent was removed under reduced pressure, and co-evaporated 3 times with ethanol to azeotrope any residual pyridine. The residue was dissolved in chloroform (20 mL) and then washed with 0.5 M, pH=7.0 potassium phosphate (20 mL). The aqueous phase was extracted 3 times with chloroform. The combined chloroform phase was washed with distilled water (3×30 mL), and dried over anhydrous sodium sulfate. The drying agent was removed by filtration. The filtrate was concentrated to give a yellow solid (109 mg) which was used for next step without further purification. The resulting 9-N-(2-butyrothiolactone)amido-11-Δ⁹-THC (16 mg, 0.0356 mmol) was dissolved in 0.36 mL of DMF/water (7/3, v/v). Potassium hydroxide aqueous solution (0.12 mL, 1 N) was added, and the solution was allowed to stand at room temperature for 10 min. Then 0.12 mL of hydrochloric acid (1 N) was added to the reaction mixture. The solution was used immediately to perform the conjugation step. The resulting hapten (0.3 mL) was added to bromo-BSA (0.75 mL, 28 mg/mL) and the reaction mixture was allowed to stand at room temperature for 3 hrs. The solution was dialyzed against PBS for 48 hrs.

### EXAMPLE 8

### Antibody Production

Antibodies can be raised to any of the haptens or compounds described herein. The antibodies can be either polyclonal or monoclonal. Standard techniques of generating antibodies will function in the invention.

For example, the generation monoclonal antibodies against a compound of the invention, involves the following steps. Following a known immunization protocol, mice are immunized with the selected compound. After an appropriate incubation period, the mouse spleen cells are harvested and fused with myeloma cells, giving rise to lymphocyte hybridomas. The hybridomas are cultured and then screened for production of antibodies that hybridize with the selected compound.

To determine the likelihood of the synthesized antigens stimulating the production of high affinity antibodies (which could be used in immunoassays to test for the presence of THC parent compounds and their metabolites), the antigens of the present invention were evaluated, prior to injection into host animals. A control Δ⁸-THC antibody (AF9-14FR-4-2, available commercially from several sources) was used for these evaluations. The Δ⁸-THC antigen, to which AF9-14FR-4-2 was raised, and Δ⁹-THC antigen were used as controls. The binding of the antigens to the antibody were evaluated by measuring the A₄₅₀.

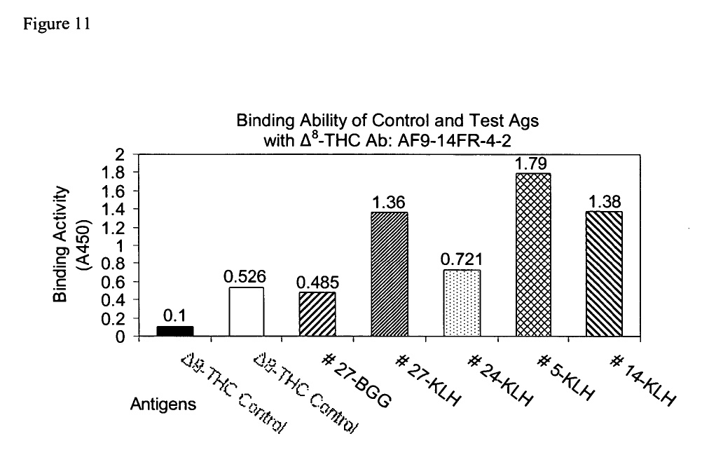

With reference to FIG. 11, the first bar indicates that very little of Δ⁹-THC was bound by the Δ⁸-THC antibody, while Δ⁸-THC antigen had a binding affinity of 0.526 (bar 2). The #27-BGG antigen (bar 3) (see FIG. 6 for structure) had a similar binding affinity for the Δ⁸-THC antibody as the Δ⁸-THC control (bar 2). The four remaining Δ⁹-THC antigens, #27-KLH (bar 4), #24-KLH (bar 5), #5-KLH (bar 6) and #14-KLH (bar 7) had a higher binding affinity for the Δ⁸-THC antibody than the Δ⁸-THC control antigen, to which the Δ⁸-THC antibody was raised. The #27-KLH antigen (bar 4) had a 2,6-fold higher binding affinity for the antibody than the control antigen (bar 2). Also, the #5-KLH (bar 6) and #14-KLH (bar 7) antigens had 3,4-fold and 2,6-fold higher affinities, respectively, for the antibody. These results also indicated that immunogens 5, 14, 24, 27, 43, 45, and 46 exhibit strong binding affinity with THC-related antibodies selected from antibody libraries.

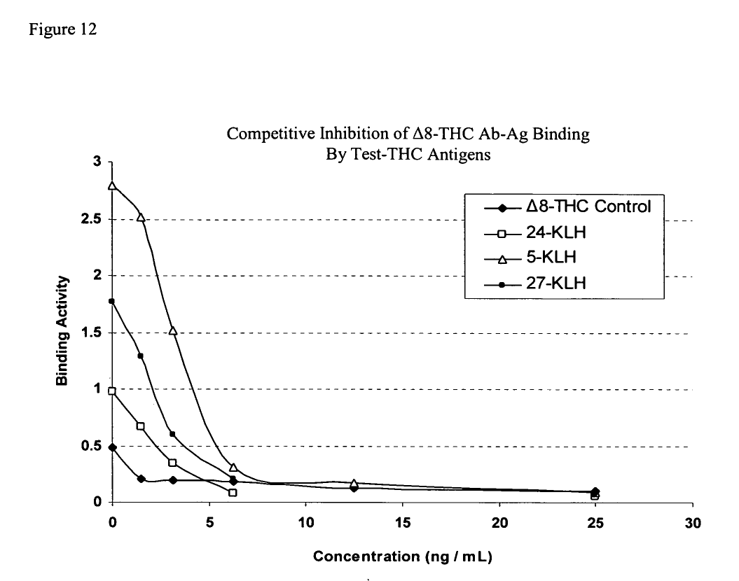

The ability of the Δ⁹-THC antigens of the present invention to compete for binding the Δ⁸-THC antibody (against the control Δ⁸-THC antigen) was also evaluated. With reference to FIG. 12, Δ⁹-THC antigens of the present invention (#24-KLH, #5-KLH and #27-KLH) bind much more strongly to the antibody than even the control antigen.

These results illustrate that antibodies raised against the antigens of the present invention will bind strongly to the Δ⁸- and Δ⁹-THC parent compounds found in saliva, as well as metabolites found in blood or urine.

Test Devices

A further aspect of the present invention is an immunoassay test device for testing a biological sample for the presence of Δ⁸, Δ⁹THC and their metabolites, having antibodies against a compound according to a compound of the present invention. In one embodiment the device is a lateral flow immunoassay device.

In general, a lateral flow immunoassay follows either a sandwich assay or competitive assay formats, which have been described and are well known to those in the art. In the following discussion strips of test strip material will be described by way of illustration and not limitation.

Generally, test strips of a test device of the present invention include a sample application zone and a detection zone. The detection zone can include one or more analytes (natural or synthetic), which are bound by a specific binding molecule, and one or more control zones. The test strip can also contain a reagent zone. The test strip can include both bibulous and non-bibulous material.

One or more specific binding specific binding molecules in the detection zone can be present throughout the thickness of the bibulous or non-bibulous material in the detection zone (for example, in one embodiment specific binding members for one or more analytes can be impregnated throughout the thickness of the test strip material in one or more analyte detection zones, and specific binding members for one or more control analytes can be impregnated throughout the thickness of the test strip material in one or more control zones). Such impregnation can enhance the extent to which the immobilized reagent can capture an analyte present in the migrating sample or specimen. Alternatively, reagents, including specific binding members and components of signal producing systems may be applied to the surface of the bibulous or non-bibulous material. Impregnation of specific binding members into test strip materials or application of specific binding members onto test strip materials may be done manually or by machine.

Nitrocellulose has the advantage that a specific binding member in the test results determination zone can be immobilized without prior chemical treatment. If the porous solid phase material comprises paper, for example, the immobilization of the antibody in the test results determination zone can be performed by chemical coupling using, for example, CNBr, carbonyldiimidazole, or tresyl chloride.

Following the application of a specific binding member, such as an antibody against the analyte (antigen) of interest, to the test results determination zone, the remainder of the porous solid phase material may be treated to block any remaining binding sites elsewhere. Blocking can be achieved by treatment with protein (for example bovine serum albumin or milk protein), or with polyvinylalcohol or ethanolamine, or any combination of these agents. A labeled reagent for the reagent zone can then be dispensed onto the dry carrier and will become mobile in the carrier when in the moist state. Between each of these various process steps (sensitization, application of unlabeled reagent, blocking and application of labeled reagent), the porous solid phase material should be dried.

To assist the free mobility of the labeled reagent when the test strip is moistened with the sample or specimen, the labeled reagent can be applied to the bibulous or non-bibulous material as a surface layer, rather than being impregnated in the thickness of the bibulous material. This can minimize interaction between the bibulous or non-bibulous material and the labeled reagent. For example, the bibulous or non-bibulous material can be pre-treated with a glazing material in the region to which the labeled reagent is to be applied. Glazing can be achieved, for example, by depositing an aqueous sugar or cellulose solution, for example of sucrose or lactose, on the carrier at the relevant portion, and drying (see, U.S. Pat. No. 5,656,503 to May et al., issued Aug. 12, 1997). The labeled reagent can then be applied to the glazed portion. The remainder of the carrier material should not be glazed.

The reagents can be applied to the carrier material in a variety of ways. Various “printing” techniques have previously been proposed for application of liquid reagents to carriers, for example micro-syringes, pens using metered pumps, direct printing and ink-jet printing, and any of these techniques can be used in the present context. To facilitate manufacture, the carrier (for example sheet) can be treated with the reagents and then subdivided into smaller portions (for example small narrow strips each embodying the required reagent-containing zones) to provide a plurality of identical carrier units.

In aspects where the analyte is detected by a signal producing system, such as by one or more enzymes that specifically react with the analyte, one or more components of the signal producing system can be bound to the analyte detection zone of the test strip material in the same manner as specific binding members are bound to the test strip material, as described above. Alternatively or in addition, components of the signal producing system that are included in the sample application zone, the reagent zone, or the analyte detection zone of the test strip, or that are included throughout the test strip, may be impregnated into one or more materials of the test strip. This can be achieved either by surface application of solutions of such components or by immersion of the one or more test strip materials into solutions of such components. Following one or more applications or one or more immersions, the test strip material is dried. Alternatively or in addition, components of the signal producing system that are included in the sample application zone, the reagent zone, or the analyte detection zone of the test strip, or that are included throughout the test strip, may be applied to the surface of one or more test strip materials of the test strip as was described for labeled reagents.

Sample Application Zone

The sample application zone is an area of a test strip where a sample, such as a fluid sample, such as a biological fluid sample such as blood, serum, saliva, or urine, or a fluid derived from a biological sample, such as a throat or genital swab, is applied. The sample application zone can include a bibulous or non-bibulous material, such as filter paper, nitrocellulose, glass fibers, polyester or other appropriate materials. One or more materials of the sample application zone may perform a filtering function, such that large particles or cells are prevented from moving through the test strip. The sample application zone can be in direct or indirect fluid communication with the remainder of the test strip, including the test results determination zone. The direct or indirect fluid communication can be, for example, end-to-end communication, overlap communication, or overlap or end-to-end communication that involves another element, such as a fluid communication structure such as filter paper such as disclosed and depicted in U.S. patent application Ser. No. 09/860408.

The sample application zone can also include compounds or molecules that may be necessary or desirable for optimal performance of the test, for example, buffers, stabilizers, surfactants, salts, reducing agents, or enzymes.

Reagent Zone

The test strip can also include a reagent zone where reagents useful in the detection of an analyte can be provided immobilized (covalent or non-covalent immobilization) or in a mobile form. The reagent zone can be on a reagent pad, a separate segment of bibulous or non-bibulous material included on the test strip, or it can be a region of a bibulous or non-bibulous material of a test strip that also includes other zones, such as an analyte detection zone. In one aspect of the invention, the reagent zone can include a labeled specific binding member, such as antibodies or active fragments thereof attached or linked to a label. Such labeled specific binding members can be made using methods known in the art. The specific binding members can bind an analyte and/or can bind a control compound.

In one aspect of the present invention, a sandwich immunoassay of Δ⁹-THC in saliva, the reagent zone includes a labeled Δ⁹-THC antibody, generated against one of the present compounds described herein. The label attached to the antibody may be of any type known in the art, such as a metal sol, a colored latex bead, a water-soluble conjugated dye, or the like. If a Δ⁹-THC is present in an applied saliva sample, the labeled antibody would bind to it, as the sample flowed down stream on the test strip. A second Δ⁹-THC antibody, this time in the unlabeled state, would be immobilized on the test line. The second antibody would also capture the Δ⁹-THC (already bound to the first Δ⁹-THC antibody) and thus create a sandwich on the test line. As the population of antibody-antigen sandwiches built up on the test line, a colored line would begin to appear, due to the concentration of the label. Excess unbound labeled Δ⁹-THC antibody (1^{st} antibody) would continue down stream. Optionally, a third antibody, capable of capturing the 1^{st} antibody can be immobilized on the control line. For example, if the 1^{st} antibody is a mouse antibody, the 3^{rd} antibody could be a goat anti-mouse antibody, which is capable of capturing most mouse antibodies. This arrangement would cause a build-up of label on the control line, indicating that the test was conducted correctly.

In a preferred embodiment of the invention, a competitive immunoassay for Δ⁹-THC in saliva, the reagent zone includes Δ⁹-THC or a Δ⁹-THC analog bound to a label. As described above, the label may be a metal sol, a colored latex particle, or a water-soluble conjugated dye, or the like. In this case, the Δ⁹-THC in the sample competes with the labeled Δ⁹-THC or Δ⁹-THC analog provided in the reagent zone for binding to a Δ⁹-THC antibody in the test results determination zone. A reduced visual signal in comparison with a control sample lacking analyte indicates the presence of Δ⁹-THC in the sample. Analogs are chemical compounds that are structurally similar to one another but differ slightly in composition, for example, in the replacement of one atom by an atom of a different element, or the presence of a particular functional group or a different chemical group.

Optionally, the test strip can be designed to assay for several different analytes at the same time. In this case, the reagent zone may include additional antibodies for the detection of other drugs of abuse, such as cocaine, barbiturates, methamphetamines, and the like, which might also be present in the sample.

The labels used in the assay can be non-particulate, water-soluble labels, or particulate labels, such as gold, or polymeric beads, such as colored beads, or particles of carbon black. Other useful labels include, for example, enzymes, chromophores or fluorophores such as they are known in the art, particularly in immunoassays, or later developed. The populations of beads are provided in powdered form on the reagent zone, which can include a bibulous material, such as filter paper, glass fibers, nylon, or nitrocellulose. These reagents are reversibly bound to the reagent zone because they can be mobilized when placed in contact with a fluid, such as a fluid sample passing along a test strip.

In another aspect of the invention, the reagent zone can include components of a signal producing system, for example, catalysts, such as enzymes, cofactors, electron donors or acceptors, and/or indicator compounds.

The reagent zone can also include compounds or molecules that may be necessary or desirable for optimal performance of the test, for example, buffers, stabilizers, surfactants, salts, reducing agents, or enzymes.

Test Results Determination Zone

In lateral flow immunoassay test strips, generally the test results determination zone includes immobilized or mobile reagents that can detect the presence of the analyte being tested for. For example, the test results zone of a Δ⁹-THC test device would have an unlabeled Δ⁹-THC antibody on the test line. Such reagents are preferably in a dry state and can be covalently immobilized, non-covalently immobilized, or not immobilized in a fluid state. The test result determination zone can include either or both of one or more analyte detection zones and one or more control zones.

Depending on the particular format and analyte being tested for, a variety of reagents can be provided at the test results determination zone. For example, the test results determination zone can include specific binding members such as antibodies, enzymes, enzymatic substrates, coenzymes, enhancers, second enzymes, activators, cofactors, inhibitors, scavengers, metal ions, and the like. One or more of the reagents provided at the test results determination zone can be bound to the test strip material. Test strips including such reagents are known in the art and can be adapted to the test device of the present invention.

In a preferred embodiment of the present invention, the one or more analyte detection zones of the test results determination zone include one or more immobilized (covalently or non-covalently immobilized) specific binding members that bind with one or more analytes of interest, such as one or more drugs, hormones, antibodies, metabolites, or infectious agents, when the analytes are also bound by specific binding members bound to a label as are provided in the reagent zone. Thus, in embodiments where the reagent zone contains one or more specific binding members for the analyte, the specific binding members of the reagent zone and analyte detection zone should bind with different epitopes on the analyte being tested for. For example, when a labeled specific binding member in the reagent zone binds with the beta-chain of hCG, then the immobilized specific binding member in the analyte detection zone should bind with another area of hCG, such as the alpha-chain of hCG. Thus, when hCG is present in the sample, the hCG will bind the labeled anti-beta hCG which carried along to the test result determination zone at the analyte detection zone which binds with the immbolized anti-alpha hCG to provide a visual readout at that locus.

The analyte detection zone can include substrates which change in an optical property (such as color, chemiluminescence or fluorescence) when an analyte is present. Such substrates are known in the art, such as, but not limited to, 1,2-phenylenediamine, 5-aminosalicylic acid, 3,3′,5,5′tetramethylbenzidine, or tolidine for peroxidase; 5-bromo-4-chloror-3-indolyl phosphate/nitroblue tetrazolium for alkaline phosphatase and 5-bromo-4-chloro-3-indolyl-beta-D-galactopyranoside, o-nitrophenyl-beta-D-galactopyranoside, napthol-AS-BI-beta-D-galactopyranoside, and 4-methyl-umbelliferyl-beta-D-galactopyranoside for beta galactosidase.

In embodiments where an analyte is detected by a signal producing system, one or more components of the signal producing system, such as enzymes, substrates, and/or indicators, can be provided in the analyte detection zone. Alternatively, the components of the signal producing system can be provided elsewhere in the test strip and can migrate to the analyte detection zone.

Optionally, the test results determination zone can include a control zone. The control zone can be upstream from, downstream from, or integral with the analyte detection zone of the test result determination zone. The control zone provides a result that indicates that the test on the test strip has performed correctly. In one preferred aspect of the present invention, the reagent zone includes a specific binding member that binds with a known analyte different from the analyte being tested for. For example, a rabbit-IgG may be provided in the reagent zone. The control zone can include immobilized (covalently or non-covalently) anti-rabbit-IgG antibody. In operation, when the labeled rabbit-IgG in the reagent zone is carried to the test result determination zone and the control zone therein, the labeled rabbit-IgG will bind with the immobilized an anti-rabbit-IgG and form a detectable signal.

The control zone can include substrates which change in an optical property (such as color, chemiluminescence or fluorescence) when a control substance is present.

The invention illustratively described herein may be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by various embodiments and optional features, modification and variation of the concepts herein disclosed may be resorted to by those skilled in the art, and that such modifications and variations are considered to be within the scope of this invention as defined by the appended claims.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

1. A compound having the chemical formula: 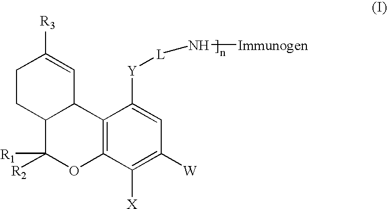
wherein
R₁, R₂, and R₃ are independently selected from the group consisting of: H, C₁₋₃ alkyl, C₁₋₃ alkoxy, C₁₋₃ thioalkyl, CN, COOH, CH₂OH, and NO₂;
Y is selected from the group consisting of: S, O, C═O, —CH₂—, —CH₂NH—, and C═N;
W is selected from the group consisting of: C₂₋₈ branched or straight chain alkyl;
X is H or C₁₋₃ alkyl;
n is an integer of from 1 to 100;
L is a linker molecule selected from the group consisting of:
CH₂(CH₂)_{1,3-5}CO, (CH₂)₀₋₅CH═CHCO, CH₂(C₆H₄)_{z}CO, COCH₂(CH₂₋₅CO, COCH₂(CH₂)₀₋₅OCH₂(CH₂)₀₋₅CO; CH₂(CH₂)₀₋₅NHCO(CH₂)₀₋₅S;
z indicates an ortho-, meta-, or para-substitution;
provided that,
when Y is O, then L is not CH₂(CH₂)_{0,2}CO; and
when Y is O and Immunogen is human serum albumin, then L is not COCH₂ (CH₂)CO;
when L is COCH₂CH₂CO, then Immunogen is not human serum albumin.

2. A compound of claim 1 having the chemical formula: 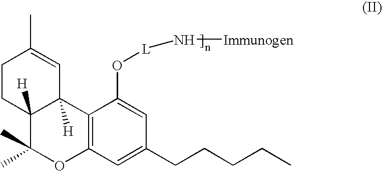
wherein
L is a linker molecule selected from the group consisting of:
CH₂(CH₂)_{1,3-5}CO, (CH₂)₀₋₅CH═CHCO, CH₂(C₆H₄)_{z}CO, COCH₂(CH₂)₂₋₅CO, COCH₂(CH₂)₀₋₅OCH₂(CH₂)₀₋₅CO, CH₂(CH₂)₀₋₅NHCO(CH₂)₀₋₅S;
n is an integer from 1 to 100;
z indicates an ortho-, meta-, or para-substitution;
Immunogen is selected from the group consisting of: Keyhold limpet hemocyanin (KLH), bovine gamma globulin (BGG), bovine serum albumin (BSA), bovine thyroglobulin (BTG), hen egg-white lysozyme (HEL), ovalbumin (VA), sperm whale myoglobin (SWM), tetranus toxoid (TT), flagellin, human IgG, and an agarose particle.

3. A compound of claim 2 wherein L is selected from the group consisting of: CH₂(CH₂)_{1,3}CO, (CH₂)₁₋₂CH═CHCO, CH₂(C₆H₄)_{z}CO, COCH₂(CH₂)₂₋₅CO, COCH₂(CH₂)₁₋₂OCH₂(CH₂)₁₋₂CO, and CH₂(CH₂)₁₋₃NHCO(CH₂)₁₋₃S;
n is an integer of from 10 to 80;
wherein Immunogen is selected from the group consisting of: Keyhold limpet hemocyanin (KLH), bovine gamma globulin (BGG), bovine serum albumin (BSA), bovine thyroglobulin (BTG), hen egg-white lysozyme (HEL), ovalbumin (VA), sperm whate myoglobin (SWM), tetranus toxoid (TT), flagelin, human IgG, and an agarose particle.

4. A compound according to claim 3 selected from the group consisting of:

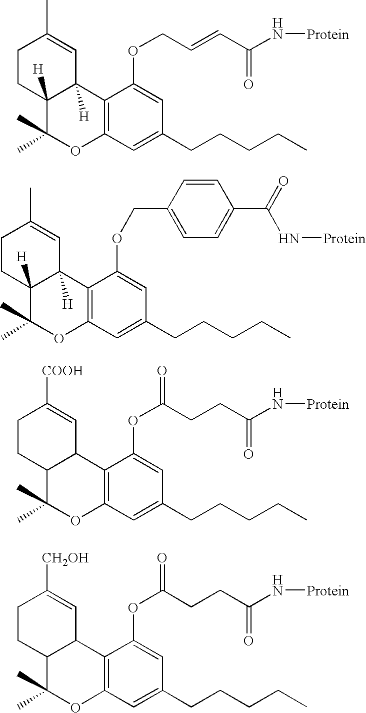

5. A method of producing antibodies comprising:
administering to a host animal a compound of the formula: 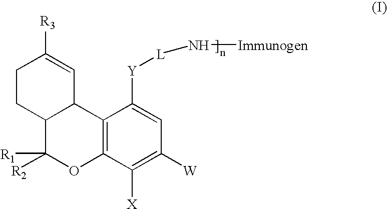
wherein
R₁, R₂, and R₃ are independently selected from the group consisting of: H, C₁₋₃ alkyl, C₁₋₃ alkoxy, C₁₋₃ thioalkyl, CN, COOH, CH₂OH, and NO₂;
Y is selected from the group consisting of: O, S, C═O, —CH₂—, —CH₂NH—, and C═N;
W is selected from the group consisting of: C₂₋₈ branched or straight chain alkyl;
X is H or C₁₋₃ alkyl;
L is a linker molecule selected from the group consisting of:
CH₂(CH₂)₁₋₅CO, (CH₂)₀₋₅CH═CHCO, CH₂(C₆H₄)_{z}CO, COCH₂(CH₂)_{0,2-5}CO, COCH₂(CH₂)₀₋₅OCH₂(CH₂)₀₋₅CO; CH₂(CH₂) ₀₋₅NHCO(CH₂)₀₋₅S;
n is an integer of from 1 to 100;
z indicates an ortho-, meta-, or para-substitution; and producing the antibodies.

6. A method of claim 5 wherein the compound has the chemical formula: 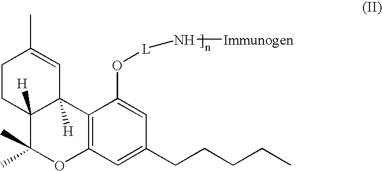
wherein
L is a linker molecule selected from the group consisting of:
CH₂(CH₂)₁₋₅CO, (CH₂)₀₋₅CH═CHCO, CH₂(C₆H₄)_{z}CO, COCH₂(CH₂)_{0,2-5}CO, COCH₂(CH₂)₀₋₅OCH₂(CH₂)ₘCO; CH₂(CH₂)₀₋₅NHCO(CH₂)₀₋₅S
n is an integer from 1 to 100;
z indicates an ortho-, meta-, or para-substitution;
Immunogen is selected from the group consisting of: Keyhold limpet hemocyanin (KLH), bovine gamma globulin (BGG), bovine serum albumin (BSA), bovine thyroglobulin (BTG), hen egg-white lysozyme (HEL), ovalbumin (VA), sperm whate myoglobin (SWM), tetranus toxoid (TT), flagellin, human IgG, and an agarose particle.

7. A specific binding molecule produced by the method of claim 6.

8. A method of claim 6 wherein L is selected from the group consisting of:
CH₂(CH₂)₁₋₃CO, (CH₂)₁₋₂CH═CHCO, CH₂(C₆H₄)_{z}CO, COCH₂(CH₂)_{0,2-5}CO, COCH₂(CH₂)₁₋₂OCH₂(CH₂)₁₋₂CO; CH₂(CH₂)₁₋₃NHCO(CH₂)₁₋₃S;
n is an integer of from 10 to 80;
Immunogen is selected from the group consisting of: Keyhold limpet hemocyanin (KLH), bovine gamma globulin (BGG), bovine serum albumin (BSA), bovine thyroglobulin (BTG), hen egg-white lysozyme (HEL), ovalbumin (VA), sperm whate myoglobin (SWM), tetranus toxoid (TT), flagellin, human IgG, and an agarose particle.

9. A method of claim 8 wherein the animal is selected from the group consisting of: a mouse, rat, goat, sheep, cow, or horse.

10. The method of claim 8 further comprising purifying the antibodies.

11. A method of claim 8 wherein the compound is selected from the group consisting of:

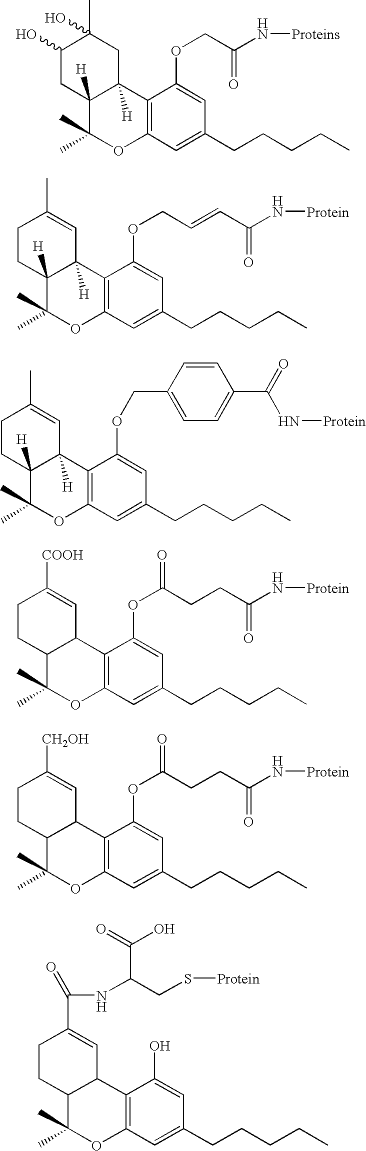

12. A compound having the formula: 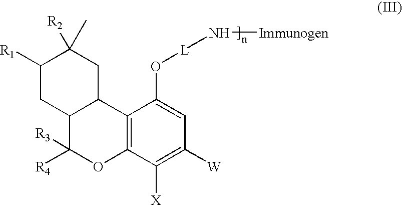
wherein
R₁ and R₂ are independently selected from the group consisting of: H, OH, NH₂, O-alkyl, COOH, CONH₂, CN, SH, and S-alkyl;
R₃ and R₄ are independently selected from the group consisting of: OH, H, NH₂, O-alkyl, COOH, CONH₂, CN, SH, C₁₋₃ alkyl, C₂₋₃ alkenyl, and S-alkyl;
X is H or C₁₋₃ alkyl;
W is C₂₋₈ branched or straight chain alkyl;
L is a linker molecule selected from the group consisting of:
CH₂(CH₂)₀₋₅CO, (CH₂)₀₋₅CH═CHCO, CH₂(C₆H₄)_{z}CO, COCH₂(CH₂)₀₋₅CO, COCH₂(CH₂)₀₋₅OCH₂(CH₂)₀₋₅CO; CH₂(CH₂) o₅NHCO(CH₂)₀₋₅S
n is an integer from 1 to 100;
z indicates an ortho-, meta-, or para-substitution.

13. A compound of according to claim 12 wherein:
R₁ and R₂ are independently selected from the group consisting of: OH, H, NH₂, O-alkyl, and SH;
R₃ and R₄ are independently selected from the group consisting of: OH, H, NH₂, O-alkyl, C₁₋₃ alkyl, C₂₋₃ alkenyl, and SH;
L is a linker molecule selected from the group consisting of:
CH₂(CH₂)₀₋₃CO, (CH₂)₁₋₂CH═CHCO, CH₂(C₆H₄)ₓCO, COCH₂(CH₂)₁₋₅CO, COCH₂(CH₂)₁₋₂OCH₂(CH₂)₁₋₂CO; CH₂(CH₂)₁₋₃NHCO(CH₂)₁₋₃S.

14. A compound of claim 12 having the structure: 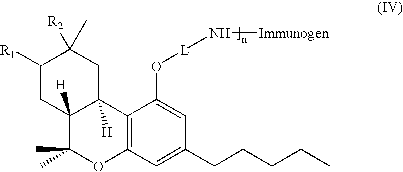
wherein
R₁ and R₂ are selected from the group consisting of: OH, H, NH₂, O-alkyl, and SH;
L is a linker molecule selected from the group consisting of:
CH₂(CH₂)₀₋₃CO, (CH₂)₁₋₂CH═CHCO, CH₂(C₆H₄)ₓCO, COCH₂(CH₂)₁₋₅CO, COCH₂(CH₂)₁₋₂OCH₂(CH₂)₁₋₂CO; CH₂(CH₂)₁₋₃NHCO(CH₂)₁₋₃S;
the immunogen is selected from the group consisting of: Keyhold limpet hemocyanin (KLH), bovine gamma globulin (BGG), bovine serum albumin (BSA), bovine thyroglobulin (BTG), hen egg-white lysozyme (HEL), ovalbumin (VA), sperm whate myoglobin (SWM), tetranus toxoid (TT), flagelin, human IgG, and an agarose particle.

15. A compound of claim 14 wherein R₁ and R₂ are each OH.

16. A compound of claim 15 having the formula:

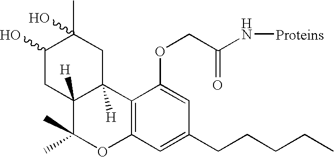

17. A method of producing antibodies comprising: administering to a mammal a composition of the formula: 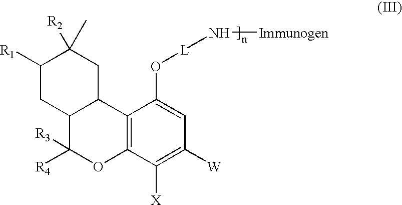
wherein
R₁ and R₂ are independently selected from the group consisting of: OH, H, NH₂, O-alkyl, COOH, CONH₂, CN, SH, and S-alkyl;
R₃ and R₄ are independently selected from the group consisting of: H, O-alkyl, CONH₂, CN, C₁₋₃ alkyl, and C₂₋₃ alkenyl;
L is a linker molecule selected from the group consisting of:
CH₂(CH₂)₀₋₅CO, (CH₂)₀₋₅CH═CHCO, CH₂(C₆H₄)_{z}CO, COCH₂(CH₂)₀₅₋CO, COCH₂(CH₂)₀₋₅OCH₂(CH₂)₀₋₅CO; CH₂(CH₂)₀₋₅NHCO(CH₂)₀₋₅S
n is an integer from 1 to 100;
z indicates an ortho-, meta-, or para-substitution.
producing the antibodies.

18. The method of claim 17 wherein:
R₁ and R₂ are independently selected from the group consisting of: OH, H, NH₂, O-alkyl, and SH;
R₃ and R₄ are independently selected from the group consisting of: H, C₁₋₃ alkyl, C₂₋₃ alkenyl, O-alkyl, CONH₂, and CN;
L is a linker molecule selected from the group consisting of:
CH₂(CH₂)₀₋₃CO, (CH₂)₁₋₂CH═CHCO, CH₂(C₆H₄)ₓCO, COCH₂(CH₂)₁₋₅CO, COCH₂(CH₂)₁₋₂OCH₂(CH₂)₁₋₂CO; CH₂(CH₂)₁₋₃NHCO(CH₂)₁₋₃S; and
the antibodies bind specifically to Δ⁹-Tetrahydrocannabinol, a Δ⁹-Tetrahydrocannabinoid, Δ⁸-Tetrahydrocannabinol, and a Δ⁸-Tetrahydrocannabinoid.

19. The method of claim 17 wherein the compound has the structure: 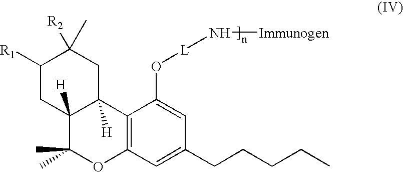
wherein
R₁ and R₂ are selected from the group consisting of: OH, H, NH₂, O-alkyl, and SH;
L is a linker molecule selected from the group consisting of:
CH₂(CH₂)₀₋₃CO, (CH₂)₁₋₂CH═CHCO, CH₂(C₆H₄)ₓCO, COCH₂(CH₂)₁₋₅CO, COCH₂(CH₂)₁₋₂OCH₂(CH₂)₁₋₂CO; CH₂(CH₂)₁₋₃NHCO(CH₂)₁₋₃S; and
the immunogen is selected from the group consisting of: Keyhold limpet hemocyanin (KLH), bovine gamma globulin (BGG), bovine serum albumin (BSA), bovine thyroglobulin (BTG), hen egg-white lysozyme (HEL), ovalbumin (VA), sperm whate myoglobin (SWM), tetranus toxoid (TT), flagelin, human IgG, and an agarose particle.

20. A method of claim 19 wherein R₁ and R₂ are each OH.

21. A specific binding molecule produced by the method of claim 20.

22. The method of claim 20 wherein the compound is selected from the group consisting of:

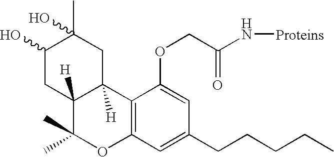

23. A compound having the formula: 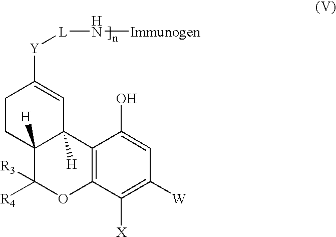
Y is selected from the group consisting of: C═O, —CH₂—, —CH₂NH—, and C═N;
L is a linker molecule selected from the group consisting of:
null, NHCH₂ (CH₂)₀₋₃CO, O(CH₂)₁₋₃CO, NH(CH₂)₂₋₃S(CH₂)₂₋₃CO, NH(CH₂)₂₋₅NHCO(CH₂)₁₋₅CO, CO(CH₂)₁₋₅CO, (CH₂)₁₋₅CO, (CH₂)₁₋₅NHCO(CH₂)₁₋₅CO, NHCH(COOH)CH₂S, and (CH₂)S(CH₂)₁₋₅CO;
R₁ and R₂ are independently selected from the group consisting of: OH, H, NH₂, O-alkyl, COOH, CONH₂, CN, SH, and S-alkyl;
R₃ and R₄ are independently selected from the group consisting of: C₁₋₃ alkyl, C₂₋₃ alkenyl, H, O-alkyl, CONH₂, CN;
W is selected from the group consisting of: C₂₋₈ branched or straight chain alkyl;
X is H or C₁₋₃ alkyl;
n is an integer from 1 to 100.

24. A compound of claim 23 having the formula: 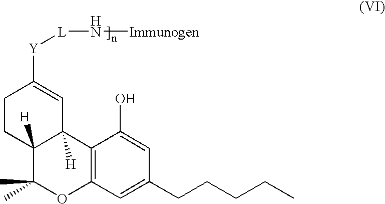
Y is selected from the group consisting of: C═O, —CH₂—, —CH₂NH—, and C═N;
L is a linker molecule selected from the group consisting of: none, NHCH₂(CH₂)₀₋₃CO, O(CH₂)₁₋₃CO, NH(CH₂)₂₋₃S(CH₂)₂₋₃CO, NH(CH₂)₂₋₅NHCO(CH₂)₁₋₅CO, CO(CH₂)₁₋₅CO, (CH₂)₁₋₅CO, (CH₂)₁₋₅CO, (CH₂)₁₋₅NHCO(CH₂)₁₋₅CO, NHCH(COOH)CH₂S, and (CH₂)S(CH₂)₁₋₅CO; and
n is an integer from 1 to 100.

25. A compound of claim 24 having the formula:

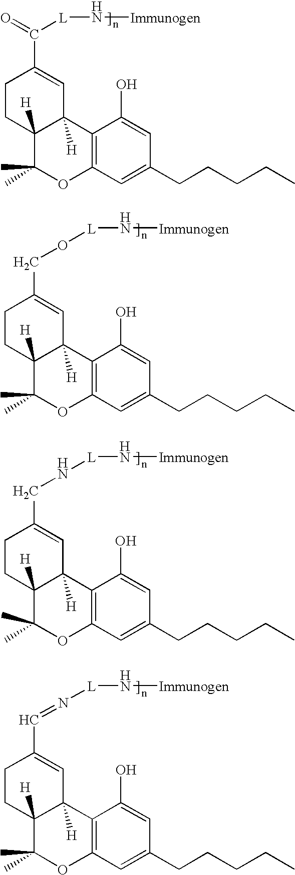

26. A method of producing antibodies comprising:
administering to a host animal a compound of the formula: 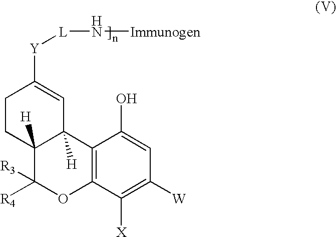
wherein
Y is selected from the group consisting of: C═O, —CH₂—, —CH₂NH—, and C═N;
L is a linker molecule selected from the group consisting of:
null, NHCH₂(CH₂)₀₋₃CO, O(CH₂)₁₋₃CO, NH(CH₂)₂₋₃S(CH₂)₂₋₃CO, NH(CH₂)₂₋₅NHCO(CH₂)₁₋₈CO, CO(CH₂)₁₋₅CO, (CH₂)₁₋₈CO, (CH₂)₁₋₅NHCO(CH₂)₁₋₈CO, NHCH(COOH)CH₂S, and (CH₂)S(CH₂)₁₋₈CO;
R₁ and R₂ are independently selected from the group consisting of: OH, H, NH₂, O-alkyl, COOH, CONH₂, CN, SH, and S-alkyl;
R₃ and R₄ are independently selected from the group consisting of: C₁₋₃ alkyl, C₂₋₃ alkenyl, H, O-alkyl, CONH₂, CN;
W is selected from the group consisting of: C₂₋₈ branched or straight chain alkyl;
X is H or C₁₋₃ alkyl; and
n is an integer from 1 to 100; and
producing the antibodies.

27. The method of claim 26 wherein the compound has the formula: 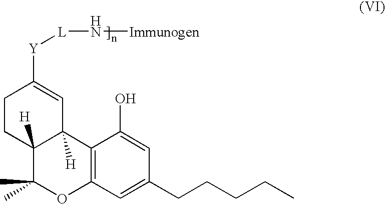
wherein
Y is selected from the group consisting of: C═O, —CH₂—, —CH₂NH—, and C═N;
L is a linker molecule selected from the group consisting of: none, NHCH₂(CH₂)₀₋₃CO, O(CH₂)₁₋₃CO, NH(CH₂)₂₋₃S(CH₂)₂₋₃CO, NH(CH₂)₂₋₅NHCO(CH₂)₁₋₅CO, CO(CH₂)₁₋₅CO, (CH₂)₁₋₅CO, (CH₂)₁₋₅NHCO(CH₂)₁₋₅CO, NHCH(COOH)CH₂S, and (CH₂)S(CH₂)₁₋₅CO; and
n is an integer from 1 to 100.

28. A specific binding molecule produced by the method of claim 27.

29. The method of claim 27 wherein the host animal is selected from the group consisting of: a mouse, a rat, a rabbit, a goat, a sheep, a cow, and a horse.

30. The method of claim 26 wherein the compound has the formula:

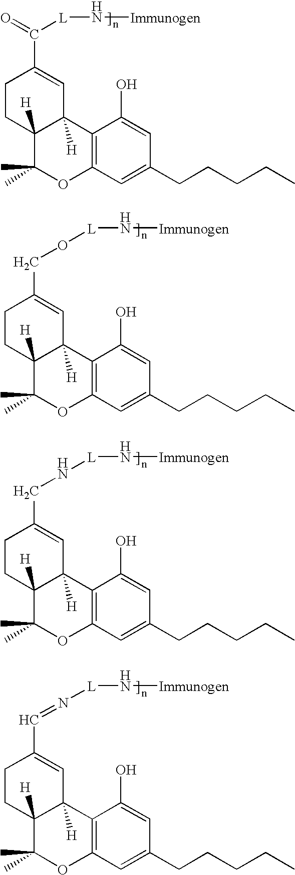

31. A compound of the formula: 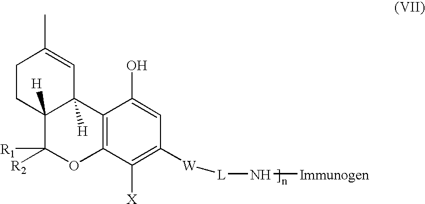
wherein
R₁ and R₂ are independently selected from the group consisting of: H, C₁₋₃ alkyl, C₁₋₃ alkoxy, C₁₋₃ thioalkyl, CN, and NO₂;
X is H or C₁₋₃ alkyl
W is selected from the group consisting of: C₂₋₈ branched or straight chain alkyl;
wherein L is selected from the group consisting of: none, CO, (CH₂)₁₋₅CO, NH(CH₂)₁₋₅CO, HNCO(CH2)₀₋₃CH₂CO, NHCO(CH₂)₁₋₃CO, O(CH₂)₁₋₃CO, OCO(CH₂)₁₋₃CO, and (CO(CH₂)₁₋₃O(CH₂)₁₋₃CO;
provided that:
if Immunogen is bovine serum albumin and W is (CH₂)₅, then L is not CO.

32. A compound of claim 31 having the formula: 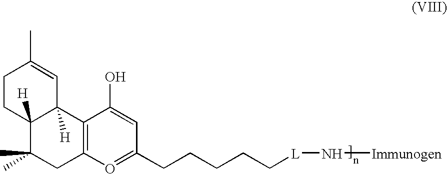
wherein L is selected from the group consisting of: none, CO, (CH₂)₁₋₅CO, NH(CH₂)₁₋₅CO, HNCO(CH2)₀₋₃CH₂CO, NHCO(CH₂)₁₋₃CO, O(CH₂)₁₋₃CO, OCO(CH₂)₁₋₃CO, and (CO(CH₂)₁₋₃O(CH₂)₁₋₃CO;
provided that:
L is not CO if Immunogen is bovine serum albumin.

33. A compound of claim 32 wherein:
Immunogen is selected from the group consisting of: Keyhold limpet hemocyanin (KLH), bovine gamma globulin (BGG), bovine serum albumin (BSA), bovine thyroglobulin (BTG), hen egg-white lysozyme (HEL), ovalbumin (VA), sperm whate myoglobin (SWM), tetranus toxoid (TT), flagelin, human IgG, and an agarose particle.

34. A compound of claim 33 wherein L is C═O.

35. A method of producing antibodies comprising:
administering to a host animal a compound of the formula: 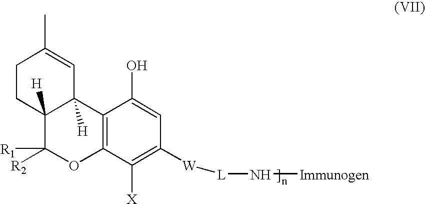
wherein
R₁ and R₂ are independently selected from the group consisting of: H, C₁₋₃ alkyl, C₁₋₃ alkoxy, C₁₋₃ thioalkyl, CN, and NO₂;
X is H or C₁₋₃ alkyl;
W is selected from the group consisting of: C₂₋₈ branched or straight chain alkyl;
wherein L is selected from the group consisting of: none, CO, (CH₂)₁₋₈CO, NH(CH₂)₁₋₈CO, HNCO(CH₂)₀₋₃CH₂CO, NHCO(CH₂)₁₋₃CO, O(CH₂)₁₋₃CO, OCO(CH₂)₁₋₃CO, and (CO(CH₂)₁₋₃O(CH₂)₁₋₃CO;
provided that:
if Immunogen is bovine serum albumin and W is (CH₂)₅, then L is not CO;
and
producing the antibodies.

36. The method of claim 35 having the formula: 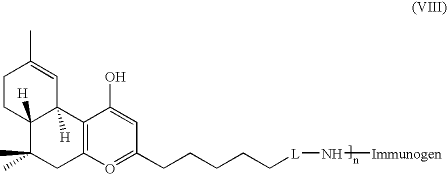
wherein L is selected from the group consisting of: none, CO, (CH₂)₁₋₅CO, NH(CH₂)₁₋₅CO, HNCO(CH2)₀₋₃CH₂CO, NHCO(CH₂)₁₋₃CO, O(CH₂)₁₋₃CO, OCO(CH₂)₁₋₃CO, and (CO(CH₂)₁₋₃O(CH₂)₁₋₃CO;
provided that:
L is not CO if Immunogen is bovine serum albumin.

37. The method of claim 36 wherein:
Immunogen is selected from the group consisting of: Keyhold limpet hemocyanin (KLH), bovine gamma globulin (BGG), bovine serum albumin (BSA), bovine thyroglobulin (BTG), hen egg-white lysozyme (HEL), ovalbumin (VA), sperm whate myoglobin (SWM), tetranus toxoid (TT), flagelin, human IgG, and an agarose particle.

38. The method of claim 37 wherein L is C═O.

39. A specific binding molecule produced by the method of claim 37.

40. The method of claim 39 wherein the host animal is selected from the group consisting of: a mouse, a rat, a rabbit, a goat, a sheep, a cow, and a horse.

41. A method of synthesizing an antigen comprising:
contacting Δ⁹-tetrahydrocannabinol with a bromoacetate to produce a 1-ether;
hydrolyzing the ester to produce a carboxylic acid derivative;
activating the carboxylic acid derivative to produce an N-succinamyl ester;
conjugating the N-succinamyl ester to synthesize an antigen.

42. The method of claim 41 wherein the activation is performed by contacting the carboxylic acid derivative with N-hydroxysuccinamide and 1-ethyl-3-(dimethylpropylamino)carbodiimide hydrochloride.

43. An antibody produced according to the method of claim 42.

44. A method of synthesizing an antigenic compound comprising the steps of: 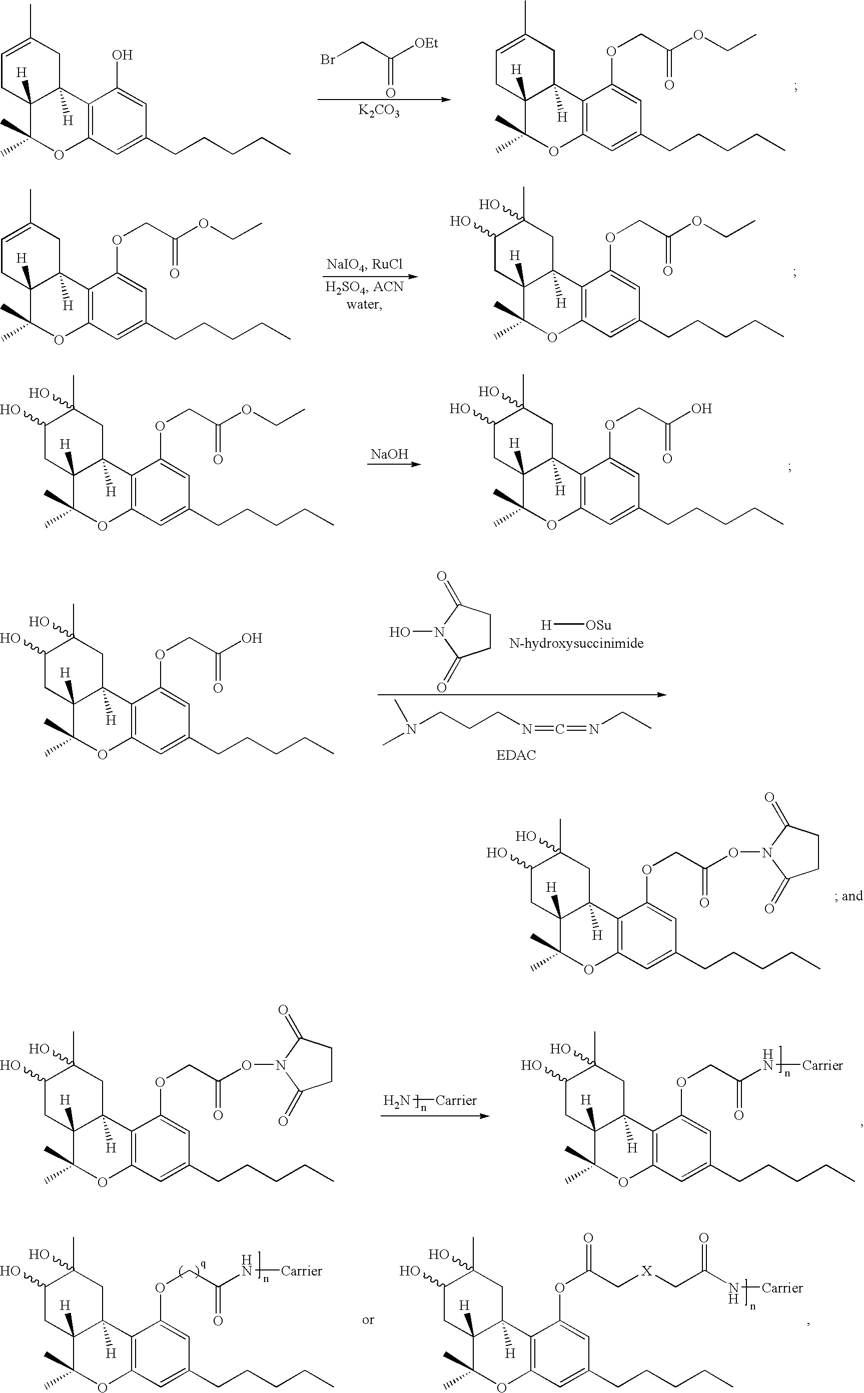
wherein q is 1-4 and X is selected from the group consisting of: none, O, or CH₂ and n is an integer from 1-100.

45. An antibody prepared according to the method of claim 44.

46. A specific binding molecule that binds to a compound selected from the group consisting of: Δ⁹-Tetrahydrocannabinol, a Δ⁹-Tetrahydrocannabinoid, Δ⁸-Tetrahydrocannabinol, and a Δ⁸-Tetrahydrocannabinoid.

47. The composition of claim 46 wherein the specific binding molecule is an antibody.

48. A cell line producing a specific binding molecule of claim 46.
